(19) United States
(12) Patent Application Publication
(10) Pub. No.: US 2009/0004706 A1
(43) Pub. Date: Jan. 1, 2009

(54) METHODS TO ENHANCE THE ACTIVITY OF LIGNOCELLULOSE-DEGRADING ENZYMES

(75) Inventors: Brian Vande Berg, Durham, NC (US); Brian Carr, Raleigh, NC (US); Nicholas B. Duck, Apex, NC (US); Michael G. Koziel, Raleigh, NC (US); Nadine Carozzi, Raleigh, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Athenix Corporation, Research Triangle Park, NC (US)

(21) Appl. No.: 12/145,201

(22) Filed: Jun. 24, 2008

Related U.S. Application Data
(63) Continuation of application No. 10/795,102, filed on Mar. 5, 2004.
(60) Provisional application No. 60/452,631, filed on Mar. 7, 2003.
(60) Provisional application No. 60/498,098, filed on Aug. 27, 2003.
(60) Provisional application No. 60/502,727, filed on Sep. 12, 2003.
(60) Provisional application No. 60/538,334, filed on Jan. 22, 2004.

(51) Int. Cl. C12P 13/04; C12P 7/56

(52) U.S. Cl. 435/128; 435/277; 435/139

(57) ABSTRACT

Methods for hydrolyzing lignocellulose are provided, comprising contacting the lignocellulose with at least one chemical treatment. Methods for pretreating a lignocellulosic material comprising contacting the material with at least one chemical are also provided. Methods for liberating a substance such as an enzyme, a pharmaceutical, or a nutraceutical from plant material are also provided. These methods are more efficient, more economical, and less toxic than current methods.

### CROSS-REFERENCE TO RELATED APPLICATIONS

This application is a continuation of U.S. application Ser. No. 10/795,102, filed Mar. 5, 2004, which claims the benefit of U.S. Provisional Application Ser. No. 60/452,631, filed Mar. 7, 2003, U.S. Provisional Application No. 60/498,098, filed Aug. 27, 2003, U.S. Provisional Application No. 60/502,727, filed Sep. 12, 2003, and U.S. Provisional Application No. 60/538,334, filed Jan. 22, 2004, the contents of which are herein incorporated by reference in their entirety.

### FIELD OF THE INVENTION

Methods to enhance the production of free sugars and oligosaccharides from plant material are provided.

### BACKGROUND OF THE INVENTION

Plant biomass is comprised of sugars and represents the greatest source of renewable hydrocarbon on earth. However, this enormous resource is under-utilized because the sugars are locked in complex polymers. These complex polymers are often referred to collectively as lignocellulose. Sugars generated from degradation of plant biomass could provide plentiful, economically competitive feedstocks for fermentation into chemicals, plastics, and fuels, including ethanol as a substitute for petroleum.

Commercial ethanol production in the U.S. is currently carried out in dry mill facilities, converting corn grain to ethanol. However corn grain is expensive, and has other high value uses, such as use in livestock feeds, and high fructose corn syrups (Wyman, ed. (1999) Handbook on Bioethanol: Production, and Utilization. Taylor & Francis, Washington, D.C., p. 1). Alternate feedstocks for ethanol production that allow production at a lower cost, and on a larger commercial scale, are desirable.

Lignocellulosics such as corn stover, which is cheap, abundant, and has no competing markets, would be preferred over grain for the production of ethanol. The limiting factor is the complex composition of the sugar polymers. Starch in corn grain is a highly branched, water-soluble polymer that is amenable to enzyme digestion. In contrast, the carbohydrates comprising lignocellulosic materials such as corn stover are more difficult to digest. These carbohydrates are principally found as complex polymers including cellulose, hemicellulose and glucans, which form the structural components of plant cell walls and woody tissues. Starch and cellulose are both polymers of glucose.

Current processes to release the sugars in lignocellulose involve many steps. A key step in the process is a harsh pretreatment. The aim of the current industry pretreatment is to increase the accessibility of cellulose to cellulose-hydrolyzing enzymes, such as the cellulase mixture derived from fermentation of the fungus Trichoderma reesei. Current pretreatment processes involve partial hydrolysis of lignocellulosic material, such as corn stover, in strong acids or bases under high temperatures and pressures. Such chemical pretreatments degrade hemicellulose and/or lignin components of lignocellulose to expose cellulose, but also create unwanted by-products such as acetic acid, furfural, and hydroxymethyl furfural. These products must be removed in additional processes to allow subsequent degradation of cellulose with enzymes or by a co-fermentation process known as simultaneous saccharification and fermentation (SSF).

The harsh conditions needed for chemical pretreatments require expensive reaction vessels, and are energy intensive. Since the chemical treatment occurs at temperature and pH conditions (for example 160° C. and 0.2% sulfuric acid at 12 atm. pressure) incompatible with known cellulosic enzymes, and produces compounds that must be removed before fermentation, this process must occur in separate reaction vessels from cellulose degradation, and must occur prior to cellulose degradation. Thus, novel methods that are more compatible with the cellulose degradation process, that do not generate toxic waste products, and that require less energy would be desirable. Further, enzymatic processes that occur in conditions similar to those used for cellulose degradation would allow development of co-treatment processes wherein the breakdown of hemicellulose and cellulose occur in the same reaction vessel, or are not separated in the manner in which current pre-treatment processes must be separated from cellulose breakdown and subsequent processes. In addition, processes that liberate sugars from lignocellulose without generating toxic products may provide additional benefits due to the increased accessibility of nutrients present in lignocellulosic material such as proteins, amino acids, lipids, and the like.

For these reasons, efficient methods are needed for conversion of lignocellulose to sugars and fermentation feedstocks.

### SUMMARY OF INVENTION

Methods are provided for hydrolyzing lignocellulose with increased efficiency without the need for a harsh pretreatment. These methods involve a chemical treatment of the lignocellulose at mild or moderate conditions to generate a treated lignocellulose, and contacting this treated lignocellulose with at least one enzyme capable of hydrolyzing a component of lignocellulose. The chemical treatment involves contacting lignocellulose with at least one chemical that acts in combination with enzyme treatment to liberate sugars.

Methods are also provided for pretreating a lignocellulosic material comprising contacting the material with at least one chemical under mild or moderate conditions to generate a treated lignocellulose. In some embodiments, the treated lignocellulose may be further treated with at least one enzyme capable of hydrolyzing lignocellulose.

Methods for liberating substances from lignocellulosic material are also encompassed. These methods comprise a chemical treatment of the lignocellulosic material under mild or moderate conditions. In some embodiments, at least one enzyme capable of hydrolyzing lignocellulose may be added subsequent to the chemical treatment. Enzymes, pharmaceuticals, and nutraceuticals may be released by treating lignocellulosic material by the methods of the invention. In some embodiments, the lignocellulosic material has been engineered to contain the substance to be released.

Chemicals for use in the above methods include oxidizing agents, denaturants, detergents, organic solvents, bases, or any combination thereof.

Methods for hydrolyzing lignocellulose comprising contacting the lignocellulose with an oxidizing agent to generate a treated lignocellulose, and contacting the treated lignocellulose with at least one enzyme capable of hydrolyzing lignocellulose are also provided.

Further provided are methods for hydrolyzing lignocellulose, comprising contacting the lignocellulose with a base at a pH of about 9.0 to about 14.0 to generate a treated lignocellulose, and contacting the treated lignocellulose with at least one enzyme capable of hydrolyzing lignocellulose.

Enzymes used in the methods of the invention can react with any component of the lignocellulose and include, but are not limited to, cellulases, xylanases, ligninases, amylases, glucuronidases, lipases, and proteases. The enzyme may be added prior to the treatment, subsequent to the treatment, or simultaneously with the chemical treatment. Further, methods that include more than one chemical treatment, either prior to or in concert with the enzyme reaction, as well as more than one enzyme treatment are provided. Multiple rounds of chemical treatment and enzyme addition are encompassed, comprising any number of treatments, in any order. The lignocellulose may be subjected to one or more physical treatments, or contact with metal ions, ozone, or ultraviolet light prior to, during, or subsequent to any treatment.

The methods of the invention may further comprise the addition of at least one fermenting organism, resulting in the production of at least one fermentation-based product. Such products include, but are not limited to, lactic acid, fuels, organic acids, industrial enzymes, pharmaceuticals, and amino acids.

### BRIEF DESCRIPTION OF THE FIGURES

FIG. 1 shows a chromatogram of sugars (glucose and xylose) that are solubilized from corn stover following H₂O₂ and cellulase treatment.

FIG. 2 shows reducing sugar content released from corn stover (measured by DNS assay) following treatment with various concentrations of hydrogen peroxide alone or in combination with enzymatic treatment.

FIG. 3 shows the percentage of hydrogen peroxide remaining after 24 hours of treatment, as well as the reducing sugar content at similar timepoints.

FIG. 4 shows the amount of microbial growth as measured by absorbance at 600 nm compared to the percentage of sugars (stover sugars or glucose and xylose) in the growth media.

### DETAILED DESCRIPTION

The present invention is drawn to several methods for hydrolyzing lignocellulose and the generation of sugars therefrom that are more economical, more efficient and less toxic than previously described treatments or pretreatments. One method involves a chemical treatment of the lignocellulose at mild or moderate treatment temperatures, pressures and/or pH ranges to form a treated lignocellulose, and contacting the treated lignocellulose with at least one enzyme capable of hydrolyzing lignocellulose.

Methods for pretreating a lignocellulosic material comprising contacting the material under mild or moderate conditions with at least one chemical are also provided. The treated lignocellulosic material may be further subjected to treatment with at least one enzyme capable of hydrolyzing lignocellulose.

Further provided are methods for liberating a substance from a lignocellulosic material comprising contacting the material with at least one chemical under mild or moderate conditions to generate a treated lignocellulosic material. The treated material may further be contacted with at least one enzyme capable of hydrolyzing lignocellulose. The lignocellulosic material may already comprise an enzyme capable of hydrolyzing lignocellulose. This lignocellulosic material comprising an enzyme may further be contacted with at least one enzyme capable of hydrolyzing lignocellulose.

In some embodiments, the plant material comprises a plant that has been genetically engineered to express at least one enzyme capable of hydrolyzing lignocellulose. In further embodiments, the plant material may be incubated under conditions that allow expression of the enzyme prior to chemical treatment. Expression of the enzyme may lead to hydrolysis of the lignocellulose prior to chemical treatment. In addition, one or more subsequent enzyme treatments may occur. Substances that may be liberated from plant material include, but are not limited to, enzymes, pharmaceuticals, and nutraceuticals. In addition, the plant material may or may not be genetically engineered to express the substance.

In any of the above methods, the chemical may be an oxidizing agent, a denaturant, a detergent, an organic solvent, a base, or any combination thereof.

In addition, methods for hydrolyzing lignocellulose comprising contacting the lignocellulose under any treatment conditions with at least one oxidizing agent to generate a treated lignocellulose, and contacting the treated lignocellulose with at least one enzyme capable of hydrolyzing lignocellulose are provided. The oxidizing agent may be a hypochlorite, hypochlorous acid, chlorine, nitric acid, a peroxyacid, peroxyacetic acid, a persulfate, a percarbonate, a permanganate, osmium tetraoxide, chromium oxide, sodium dodecylbenzenesulfonate, or a compound capable of generating oxygen radicals.

Further provided are methods for hydrolyzing lignocellulose comprising contacting the lignocellulose with a base at a pH of about 9.0 to about 14.0 to generate a treated lignocellulose, and contacting the treated lignocellulose with at least one enzyme capable of hydrolyzing lignocellulose. This method encompasses treatment conditions comprising any range of temperature or pressure. It is recognized that for this method as well as the method using an oxidizing agent that mild or moderate treatment conditions may be used.

It is recognized that the enzyme or enzymes may be added at the same time, prior to, or following the addition of the chemical solution(s). When added simultaneously, the chemical or chemical combination will be compatible with the enzymes selected for use in the treatment process. When the enzymes are added following the treatment with the chemical solution(s), the conditions (such as temperature and pH) may be altered prior to enzyme addition. In one embodiment, the pH is adjusted to be optimal for the enzyme or enzymes prior to enzyme addition. In another embodiment, the temperature is adjusted to be optimal for the enzyme or enzymes prior to enzyme addition. Multiple rounds of chemical treatments can be performed, with or without subsequent or simultaneous enzyme additions. In addition, multiple rounds of enzyme addition are also encompassed.

“Treated lignocellulose” or “treated lignocellulosic material” or “treated material” is defined as lignocellulose that has been at least partially hydrolyzed by some form of chemical or physical treatment during a ‘treatment process’ or ‘treatment’. Typically, one or more of the polymer components is hydrolyzed during the treatment so that other components are more accessible for downstream applications. Alternatively, a treatment process can alter the structure of lignocellulose so that it is more digestible by enzymes following treatment in the absence of hydrolysis. The lignocellulose may have been previously treated to release some or all of the sugars.

By “mild treatment” or “mild conditions” is intended a treatment at a temperature of about 20° C. to about 80° C., at a pressure less than about 2 atm, and a pH between about pH 5.0 and about pH 8.0. By “moderate treatment” or “moderate conditions” is intended at least one of the following conditions: a temperature of about 10° C. to about 90° C., a pressure less than about 2 atm, and a pH between about pH 4.0 and about pH 10.0. When the treatment is performed under moderate conditions, two of the three parameters may fall outside the ranges listed for moderate conditions. For example, if the temperature is about 10° C. to about 90° C., the pH and pressure may be unrestricted. If the pH is between about 4.0 and about 10.0, the temperature and pressure may be unrestricted. If the pressure is less than about 2.0 atm., the pH and temperature may be unrestricted.

By “chemical” or “chemical solution” is intended an oxidizing agent, denaturant, detergent, organic solvent, base, or any combination of these. By “oxidizing agent” is intended a substance that is capable of increasing the oxidation state of a molecule. Oxidizing agents act by accepting electrons from other molecules, becoming reduced in the process. Oxidizing agents include, but are not limited to, hydrogen peroxide, urea hydrogen peroxide, benzoyl peroxide, superoxides, potassium superoxide, hypochlorites, hypochlorous acid, chlorine, nitric acid, peroxyacids, peroxyacetic acid, persulfates, percarbonates, permanganates, osmium tetraoxide, chromium oxide, and sodium dodecylbenzenesulfonate. Oxidizing agents include peroxide-containing structures as well as compounds capable of generating oxygen radicals. By “peroxide-containing structure” is intended a compound containing the divalent ion —O—O—.

By “denaturant” is intended a compound that disrupts the structure of a protein, carbohydrate, or nucleic acid. Denaturants include hydrogen bond-disrupting agents. By “hydrogen bond-disrupting agents” or “hydrogen bond disruptor” is intended a chemical or class of chemicals known to disrupt hydrogen bonding, and/or to prevent formation of hydrogen bonds, and/or to prevent re-formation after disruption. Hydrogen bond-disrupting agents include, but are not limited to, chaotropic agents, such as urea, guanidinium hydrochloride, and amine oxides, such as N-methylmorpholine N-oxide.

By “detergent” is intended a compound that can form micelles to sequester oils. Detergents include anionic, cationic, or neutral detergents, including, but not limited to, Nonidet (N)P-40, sodium dodecyl sulfate (SDS), sulfobetaine, n-octylglucoside, deoxycholate, Triton X-100, and Tween 20. Included in the definition are surfactants. By “surfactant” is intended a compound that can lower the surface tension of water.

By “organic solvent” is intended a solution comprised in the greatest amount by a carbon-containing compound. Organic solvents include, but are not limited to, dimethyl formamide, dimethylsulfoxide, and methanol.

By “base” is intended a chemical species that donates electrons or hydroxide ions or that accepts protons. Bases include, but are not limited to, sodium carbonate, potassium hydroxide, calcium hydroxide, magnesium hydroxide, sodium hydroxide, aluminum hydroxide, lithium hydroxide, cesium hydroxide, rubidium hydroxide, barium hydroxide, strontium hydroxide, tin (II) hydroxide, and iron hydroxide.

The chemical or chemicals may be removed or diluted from the treated lignocellulose prior to enzyme addition or additional chemical treatment. This may assist in optimizing conditions for enzyme activity, or subsequent microbial growth. Alternatively, a small amount of at least one enzyme may be incubated with the treated lignocellulose, prior to contact with a larger amount of at least one enzyme. The chemical may be removed or diluted prior to addition of the larger amount of enzyme. The removal or dilution may occur by any method known in the art, including, but not limited to, washing, gravity flow, pressure, and filtration. The chemical or chemicals that are removed from the treated lignocellulose (thereby defined as a “recycled chemical”) may be reused in one or more subsequent incubations.

Further, the method may be performed one or more times in whole or in part. That is, one may perform one or more reactions with a chemical solution, or individual chemicals, followed by one or more enzyme treatment reactions. The chemicals or chemical solutions may be added in a single dose, or may be added in a series of small doses. Further, the entire process may be repeated one or more times as necessary. Therefore, one or more additional treatments with chemical or enzyme are encompassed.

The methods result in the production of soluble materials, including hydrolyzed sugars (hydrolyzate), and insoluble materials. During, or subsequent to such treatments, the liquid containing soluble materials may be removed, for example by a batch method, by a continuous method, or by a fed-batch method. The sugars may be separated from the soluble material and may be concentrated or purified. In addition, the treated lignocellulose, including the soluble materials and the residual solids may be subjected to processing prior to use. The soluble or insoluble materials may be removed or diluted, for example, with water or fermentation media, or the pH of the material may be modified. The removal or dilution may occur by any method known in the art, including, but not limited to, washing, gravity flow, pressure, and filtration. The materials may also be sterilized, for example, by filtration.

Physical treatments, such as grinding, boiling, freezing, milling, vacuum infiltration, and the like may also be used with the methods of the invention. A physical treatment such as milling allows a higher concentration of lignocellulose to be used in batch reactors. By “higher concentration” is intended up to about 20%, up to about 25%, up to about 30%, up to about 35%, up to about 40%, up to about 45%, or up to about 50% lignocellulose. The chemical and/or physical treatments can be administered concomitantly or sequentially with respect to the treatment methods of the invention. The lignocellulose may also be contacted with a metal ion, ultraviolet light, ozone, and the like. These treatments may enhance the effect of the chemical treatment for some materials by inducing hydroxyl radical formation. The methods of the invention can be carried out in any suitable container including vats, commercial containers, bioreactors, batch reactors, fermentation tanks or vessels. During the treatment of the invention, the reaction mixture may be agitated or stirred.

The methods of the invention improve the efficiency of biomass conversion to simple sugars and oligosaccharides. Efficient biomass conversion will reduce the costs of sugars that can then be converted to useful fermentation based products. By “fermentation-based product” is intended a product produced by chemical conversion or fermentation. Such products include, but are not limited to, specialty chemicals, chemical feedstocks, plastics, solvents and fuels. Specific products that may be produced by the methods of the invention include, but not limited to, biofuels (including ethanol); lactic acid; plastics; specialty chemicals; organic acids, including citric acid, succinic acid and maleic acid; solvents; animal feed supplements; pharmaceuticals; vitamins; amino acids, such as lysine, methionine, tryptophan, threonine, and aspartic acid; industrial enzymes, such as proteases, cellulases, amylases, glucanases, lactases, lipases, lyases, oxidoreductases, and transferases; and chemical feedstocks. The methods of the invention are also useful to generate feedstocks for fermentation by fermenting microorganisms. In one embodiment, the method further comprises the addition of at least one fermenting organism. By “fermenting organism” is intended an organism capable of fermentation, such as bacteria and fungi, including yeast. Such feedstocks have additional nutritive value above the nutritive value provided by the liberated sugars.

The methods of the invention are also useful for the development or modification of methods to process lignocellulosic materials. The methods are useful to modify or improve handling characteristics of lignocellulose-containing materials such as viscosity, as well as reduce feedstock bulk and particle size, which can be useful in liberation of sugars, use as a feedstock, or in preparation of the lignocellulose for use of further methods. Further, the methods of the invention can be used to reduce waste bulk, and to improve waste properties from industrial processes that generate lignocellulosic waste. Particularly the methods will be useful to reduce water content, and/or increase dryability, nutritive value or composition.

In one embodiment, the chemical treatment reduces the number of biological contaminants present in the lignocellulosic feedstock. This may result in sterilization of the feedstock. (See Example 9 in the Experimental section).

### Treatment Conditions

The enzymes are reacted with substrate under mild or moderate conditions that do not include extreme heat or acid treatment as is currently utilized for biomass conversion using bioreactors. For example, enzymes can be incubated at about 20° C. to about 80° C., preferably about 30° C. to about 65° C., more preferably about 37° C. to about 45° C., more preferably about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., in buffers of low to medium ionic strength, and neutral pH. Surprisingly the chemical treatment is capable of releasing or liberating a substantial amount of the sugars. By “substantial” amount is intended at least about 20%, about 30%, about 40%, about 50%, about 60%, about 70%, about 80%, about 85%, about 90%, about 95% and greater of available sugar.

The temperature of the chemical treatment may range from about 10° C. to about 100° C. or greater, about 10° to about 90°, about 20° C. to about 80° C., about 30° C. to about 70° C., about 40° C. to about 60° C., about 37° C. to about 50° C., preferably about 37° C. to about 100° C., more preferably about 50° C. to about 90° C., most preferably less than about 90° C., or less than about 80° C., or about 80° C. The method of the invention can be performed at many different temperatures but it is preferred that the treatment occur at the temperature best suited to the enzyme being used, or the predicted enzyme optimum of the enzymes to be used. In the absence of data on the temperature optimum, one may perform the treatment reactions at 50° C. first, then at higher or lower temperatures. Comparison of the results of the assay results from this test will allow one to modify the method to best suit the enzymes being tested. The pH of the treatment mixture may range from about pH 2.0 to about pH 14.0, but when the chemical is an oxidizing agent, denaturant, detergent, or organic solvent, the pH is preferably about 3.0 to about 7.0, more preferably about 3.0 to about 6.0, even more preferably about 3.0, about 5.0, about 3.5, about 4.0, about 4.5, or about 5.0. When the chemical is a base, the pH is preferably about pH 9.0 to about pH 14.0, more preferably about pH 10.0 to about pH 13.0, even more preferably about pH 11.0 to about pH 12.5, most preferably about pH 12.0. Again, the pH may be adjusted to maximize enzyme activity and may be adjusted with the addition of an enzyme or enzyme mixture, or prior to enzyme addition.

The final concentration of chemical may range from about 0.1% to about 10%, preferably about 0.3% to about 8%, more preferably about 0.3% to about 5.0%, or about 0.4% to about 3.0%, even more preferably, about 0.5% about 0.6%, about 0.7%, about 0.8%, about 0.9%, about 1.0%. The concentration of lignocellulose may be about 1% to about 60%, preferably about 10% to about 40%, more preferably about 20%, about 25%, about 30%, about 35%. The treatment reaction may occur from several minutes to several hours, such as for at least about 8 hours to at least about 48 hours, more preferably at least about 12 hours to at least about 36 hours, for at least about 16 hours to at least about 24 hours, for at least about 20 hours, more preferably for at least about 10 hours, most preferably for at least about 10 minutes, at least about 20 minutes, at least about 30 minutes, at least about 1 hour, at least about 1.5 hours, at least about 2.0 hours, at least about 2.5 hours, at least about 3 hours. The reaction may take place from about 0 to about 2 atm. In order to determine optimal reaction conditions (including optimal amount of chemical and substrate loads, optimal length of incubation, optimal temperature, pH, buffer, and pressure), aliquots of the mixtures can be taken at various time points before and after addition of the assay constituents, and the release of sugars can be measured by the modified DNS assay described in U.S. Application No. 60/432,750, herein incorporated by reference.

In one embodiment, the methods involve a chemical treatment of the lignocellulose at a temperature from about 0° C. to about 100° C., at a pressure less than about 2 atm., and at a pH between about pH 2.0 and about pH 14.0. In other embodiments, at least one of these conditions is sufficient for hydrolyzing lignocellulose. In still other embodiments, at least two of these conditions are sufficient for hydrolyzing lignocellulose.

In one aspect of the invention the lignocellulosic substrates or plant biomass, is degraded and converted to simple sugars and oligosaccharides for the production of ethanol or other useful products. Sugars released from biomass can be converted to useful fermentation products including but not limited to amino acids, vitamins, pharmaceuticals, animal feed supplements, specialty chemicals, chemical feedstocks, plastics or other organic polymers, lactic acid, and ethanol, including fuel ethanol.

In contrast to current methods, complex mixtures of polymeric carbohydrates and lignin, or actual lignocellulose can be used as the substrate hydrolyzed by biomass conversion enzymes. A specific assay has been developed to measure the release of sugars and oligosaccharides from these complex substrates. The assay uses any complex lignocellulosic material, including corn stover, sawdust, woodchips, and the like. In this assay the lignocellulosic material such as corn stover is incubated with enzymes(s) for various times and the released reducing sugars measured by the dinitrosalisylic acid assay as described in U.S. Provisional Application No. 60/432,750. Various additional assay methods can be used, such as those that can detect reducing sugars, to quantitate the monomeric sugars or oligomers that have been solubilized as a result of the chemical treatment. For example, high performance liquid chromatography (HPLC) methods allow for qualitative and quantitative analysis of monomeric sugars and oligomers.

The methods of the invention are also useful to generate feedstocks for fermentation. Such feedstocks have nutritive value beyond the nutritive value provided by the liberated sugars, due to the solubilization of proteins, amino acids, lignin (carbon source), lipids and minerals (including iron). As compared to other methods for the generation of feedstocks from lignocellulosic materials, this method requires little or no cleanup of the solubles prior to fermentation. Feedstocks generated in this manner may be used for the fermentation of microorganisms such as bacteria and fungi, including yeast.

The methods of the invention are also useful for the development or modification of methods to process lignocellulosic materials. As such, these methods may produce lignocellulose streams with altered compositions, lignocellulose steams with reduced viscosity, lignocellulose streams of reduced mass, as well as lignocellulose streams of reduced water content or capacity. Furthermore, the methods are suitable for the recovery of sugars from lignocellulose streams recalcitrant to hydrolysis, including agricultural waste products. The recovery would allow sugars to be reintegrated into the feedstock flow and allow waste streams to be further reduced. Additionally, the method would allow agricultural waste streams with reduced sugar contents to be generated that are more suitable as a fibrous component for incorporation into ruminant diets.

### Oxidizing Agents

The relative strengths of oxidizing agents (see, for example, http://hyperphysics.phy-astr.gsu.edu/hbase/chemical/c1) can be inferred from their standard electrode potentials (see, for example, http://hyperphysics.phy-astr.gsu.edu/hbase/chemical/c1). The strongest oxidizing agents are shown from the standard electrode table (see, for example, http://hyperphysics.phy-astr.gsu.edu/hbase/tables/c1. A partial listing of oxidizing agents includes bromates; chloric acid; chlorous acid; chlorinated isocyanurates; chromates; dichromates; halogens, including fluorine, chlorine, and bromine; hypochlorites; hypochlorous acid; nitric acid; nitrates; nitrites; oxygen; perborates; perchlorates; perchloric acid; periodates; permanganates; peroxides, including hydrogen peroxide, hydroperoxides, ketone peroxides, organic peroxides, and inorganic peroxides; peroxyacids; and persulfates.

Oxidizing and bleaching agents used in the paper industy include chlorine and chlorinated compounds; chlorine; sodium chlorate; sodium chlorite; hypochlorite; sodium hypochlorite; calcium hypochlorite; other hypochlorites; chloroidocynrates; miscellaneous chlorine compounds, 1,3-dichloro-5,5-dimethyl hydantoin (DCDMH); oxygen and oxygenated compounds; hydrogen peroxide; ozone; sodium perborate; potassium permanganate; organic peroxides; benzoyl peroxide; other organic peroxides; inorganic peroxides; sodium peroxide; calcium peroxide; magnesium peroxide; sodium percarbonate; other oxygenated compounds; peracetic and peroxymonosulfuric acid; metal oxyacids; and nitric and nitrous acids.

### Hydrogen Peroxide

Hydrogen peroxide (H₂O₂) is the protonated form of the peroxide ion (O₂²⁻); it is synthesized by oxidation process and can be purchased commercially as a dilution in water at concentrations up to 70%. Additionally, hydrogen peroxide can also be synthesized from the one-electron reduced form of oxygen (O₂^{•-}), either spontaneously or by utilization of the enzyme superoxide dismutase.

Hydrogen peroxide is a potent oxidizing agent. It is well known in the art that H₂O₂ can be reduced to the hydroxyl radical (HO^{•}) in the presence of appropriate stimulants. These stimulants include metal cations (such as Fe²⁺), ultraviolet light, and ozone. The hydroxyl radical is a very strong oxidative reagent.

While enzymes that can hydrolyze lignocellulose are too big to penetrate plant cell walls, hydrogen peroxide molecules are small enough to pass through. In the environment, hydrogen peroxide (and hydroxyl radicals) may be responsible for digestion of plant biomass that is observed following treatment with hydrogen peroxide (see, for example, Xu and Goodell (2001) J. Biotech. 87:43-57; Green and Highley (1997) Int. Biodeterioration Biodegredation 39:113-124). Other lignocellulose treatments involving hydrogen peroxide have been either carried out under alkaline conditions, or at high temperatures, or both (see, for example, Kim et al. (1996) Appl. Biochem. Biotech. 57/58:147-156; Kim et al. (2001) Appl. Biochem. Biotech. 91-93:81-94; Doner et al. (2001); Leathers et al. (1996) Appl. Biochem. Biotech. 59:334-347).

In addition to hydrogen peroxide, it is common knowledge that other compounds can generate hydroxyl radicals through various chemistries. One example is hypochlorous acid (HOCl), which can form hydroxyl radicals by reaction with electron donors such as superoxide radical (O₂.⁻) or ferrous iron (Fe²⁺).

The hydroxyl radical is one example of an oxygen radical compound that possesses oxidative properties. Other compounds that contain an oxygen radical and possess similar properties are known in the art. These compounds include the superoxide radical (O₂.⁻), singlet oxygen (¹O₂), nitric oxide (NO.), peroxyl radicals (ROO.), and alkoxyl radicals (LO.). One or more of these compounds may be useful in the processes of the invention.

Enzyme Nomenclature and Applications

The nomenclature recommendations of the IUBMB are published in Enzyme Nomenclature 1992 [Academic Press, San Diego, Calif., ISBN 0-12-227164-5 (hardback), 0-12-227165-3 (paperback)] with Supplement 1 (1993), Supplement 2 (1994), Supplement 3 (1995), Supplement 4 (1997) and Supplement 5 (in Eur. J. Biochem. (1994) 223:1-5; Eur. J. Biochem. (1995) 232:1-6; Eur. J. Biochem. (1996) 237:1-5; Eur. J. Biochem. (1997)250:1-6, and Eur. J. Biochem. (1999)264:610-650; respectively). The classifications recommended by the IUBMB are widely recognized and followed in the art. Typically, enzymes are referred to in the art by the IUBMB enzyme classification, or EC number. Lists of enzymes in each class are updated frequently, and are published by IUBMB in print and on the Internet.

Another source for enzyme nomenclature base on IUBMB classifications can be found in the ENZYME database. ENZYME is a repository of information relative to the nomenclature of enzymes. It is primarily based on the recommendations of the Nomenclature Committee of the International Union of Biochemistry and Molecular Biology (IUBMB) and it describes each type of characterized enzyme for which an EC (Enzyme Commission) number has been provided (Bairoch (2000) Nucleic Acids Res 28:304-305). The ENZYME database describes for each entry: the EC number, the recommended name, alternative names (if any), the catalytic activity, cofactors (if any), pointers to the SWISS-PROT protein sequence entries(s) that correspond to the enzyme (if any), and pointers to human disease(s) associated with a deficiency of the enzyme (if any).

“Cellulase” includes both exohydrolases and endohydrolases that are capable of recognizing and hydrolyzing cellulose, or products resulting from cellulose breakdown, as substrates. Cellulase includes mixtures of enzymes that include endoglucanases, cellobiohydrolases, glucosidases, or any of these enzymes alone, or in combination with other activities. Organisms producing a cellulose-hydrolyzing activity often produce a plethora of enzymes, with different substrate specificities. Thus, a strain identified as digesting cellulose may be described as having a cellulase, when in fact several enzyme types may contribute to the activity. For example, commercial preparations of ‘cellulase’ are often mixtures of several enzymes, such as endoglucanase, exoglucanase, and glucosidase activities.

Thus, “cellulase” includes mixtures of such enzymes, and includes commercial preparations capable of hydrolyzing cellulose, as well as culture supernatant or cell extracts exhibiting cellulose hydrolyzing activity, or acting on the breakdown products of cellulose degradation, such as cellotriose or cellobiose.

“Endoglucanase” or “1,4-β-D-glucan 4-glucanohydrolase” or “β-1, 4, endocellulase” or “endocellulase”, or “cellulase” EC 3.2.1.4 includes enzymes that cleave polymers of glucose attached by β-1,4 linkages. Substrates acted on by these enzymes include cellulose, and modified cellulose substrates such as carboxymethyl cellulose, RBB-cellulose, and the like.

“Cellobiohydrolase” or “1,4, -β-D-glucan cellobiohydrolase” or “cellulose 1,4-β-cellobiosidase” or “cellobiosidase” includes enzymes that hydrolyze 1,4-β-D-glucosidic linkages in cellulose and cellotetraose, releasing cellobiose from the non-reducing ends of the chains. Enzymes in group EC 3.2.1.91 include these enzymes.

“β-glucosidase” or “glucosidase” or “β-D-glucoside glucohydrolase” or “cellobiase” EC 3.2.1.21 includes enzymes that release glucose molecules as a product of their catalytic action. These enzymes recognize polymers of glucose, such as cellobiose (a dimer of glucose linked by β-1, 4 bonds) or cellotriose (a trimer of glucose linked by β-1,4 bonds) as substrates. Typically they hydrolyze the terminal, non-reducing β-D-glucose, with release of β-D-glucose.

TABLE 1
Cellulases include, but are not limited to, the following classes of enzymes

| Name Used in | | | | |
|---|---|---|---|---|
| this | | EC | | |
| application | EC Name | Classification | Alternate Names | Reaction catalyzed |
| 1,4-β- | Cellulase | 3.2.1.4 | Endoglucanase;. | Endohydrolysis of 1,4- |
| endoglucanase | | | Endo-1,4-β-glucanase;. | β-D-glucosidic linkages |
| | | | Carboxymethyl | |
| | | | cellulase; | |
| | | | β-1,4-endoglucanase; | |
| | | | 1,4-β-endoglucanase | |
| 1,3-β- | Endo-1,3(4)- | 3.2.1.6 | Endo-1,4-β-glucanase; | Endohydrolysis of 1,3- |
| endoglucanase | β-glucanase | | Endo-1,3-β-glucanase; | or 1,4-linkages in β-D- |
| | | | Laminarinase; | glucans when the |
| | | | 1,3-β-endoglucanase | reducing glucose |
| | | | | residue is substituted at |
| | | | | C-3 |
| β-glucosidase | β-glucosidase | 3.2.1.21 | Gentobiase; | Hydrolysis of terminal, |
| | | | Cellobiase; | non-reducing β-D- |
| | | | Amygdalase | glucose residues with |
| | | | | release of β-D-glucose |
| 1,3-1,4-β- | Licheninase | 3.2.1.73 | Lichenase; | Hydrolysis of 1,4-β-D- |
| endoglucanase | | | β-glucanase; | glycosidic linkages in β- |
| | | | Endo-β-1,3-1,4 | D-glucans containing |
| | | | glucanase; | 1,3- and 1,4-bonds |
| | | | 1,3-1,4-β-D-glucan 4- | |
| | | | glucanohydrolase; | |
| | | | Mixed linkage β- | |
| | | | glucanase; | |
| | | | 1,3-1,4-β-endoglucanase | |
| 1,3-1,4-β- | Glucan 1,4-β- | 3.2.1.74 | Exo-1,4-β-glucosidase; | Hydrolysis of 1,4- |
| exoglucanase | glucosidase | | 1,3-1,4-β-exoglucanase | linkages in 1,4-β-D- |
| | | | | glucans so as to remove |
| | | | | successive glucose units |
| Cellobiohydrolase | Cellulose 1,4- | 3.2.1.91 | Exoglucanase; | Hydrolysis of 1,4-β-D- |
| | β- | | Exocellobiohydrolase; | glucosidic linkages of |
| | cellobiosidase | | 1,4-β-cellobiohydrolase; | cellulose and |
| | | | Cellobiohydrolase | cellotetraose, releasing |
| | | | | cellobiose from the non- |
| | | | | reducing ends of the |
| | | | | chains |

“Xylanase” includes both exohydrolytic and endohydrolytic enzymes that are capable of recognizing and hydrolyzing xylan, or products resulting from xylan breakdown, as substrates. In monocots, where heteroxylans are the principal constituent of hemicellulose, a combination of endo-1,4-beta-xylanase (EC 3.2.1.8) and beta-D-xylosidase (EC 3.2.1.37) may be used to break down xylan to xylose. Additional debranching enzymes are capable of hydrolyzing other sugar components (arabinose, galactose, mannose) that are located at branch points in the xylan structure. Additional enzymes are capable of hydrolyzing bonds formed between hemicellulosic sugars (notably arabinose) and lignin.

“Endoxylanase” or “1,4-β-endoxylanase” or “1,4-β-D-xylan xylanohydrolase” or (EC 3.2.1.8) include enzymes that hydrolyze xylose polymers attached by β-1, 4 linkages. Endoxylanases can be used to hydrolyze the hemicellulose component of lignocellulose as well as purified xylan substrates.

“Exoxylanase” or “β-xylosidase” or “xylan 1,4-β-xylosidase” or “1,4-β-D-xylan xylohydrolase” or “xylobiase” or “exo-1,4-β-xylosidase” (EC 3.2.1.37) includes enzymes that hydrolyze successive D-xylose residues from the non-reducing terminus of xylan polymers.

“Arabinoxylanase” or “glucuronoarabinoxylan endo-1,4-β-xylanase” or “feraxan endoxylanase” includes enzymes that hydrolyze β-1, 4 xylosyl linkages in some xylan substrates.

TABLE 2
Xylanases include, but are not limited to, the following classes of enzymes

| Name Used in | | EC | | |
|---|---|---|---|---|
| this application | EC Name | Classification | Alternate Names | Reaction catalyzed |
| 1,4-β- | Endo-1,4-β- | 3.2.1.8 | 1,4-β-D-xylan | Endohydrolysis of 1,4-β-D- |
| endoxylanase | xylanase | | xylanohydrolase; | xylosidic linkages in xylans |
| | | | 1,4-β-endoxylanase | |
| 1,3-β- | Xylan endo-1, | 3.2.1.32 | Xylanase; | Random hydrolysis of 1,3- |
| endoxylanase | 3-β-xylosidase | | Endo-1,3-β-xylanase; | β-D-xylosidic linkages in |
| | | | 1,3 β-endoxylanase | 1,3-β-D-xylans |
| β-xylosidase | Xylan 1,4-β- | 3.2.1.37 | β-xylosidase; | Hydrolysis of 1,4-β-D- |
| | xylosidase | | 1,4-β-D-xylan | xylans removing successive |
| | | | xylohydrolase; | D-xylose residues from the |
| | | | Xylobiase; | non-reducing termini |
| | | | Exo-1,4-β-xylosidase | |
| Exo-1,3-β- | Xylan 1,3-β- | 3.2.1.72 | Exo-1,3-β-xylosidase | Hydrolysis of successive |
| xylosidase | xylosidase | | | xylose residues from the |
| | | | | non-reducing termini of 1,3- |
| | | | | β-D-xylans |
| Arabinoxylanase | Glucuronoarabinoxylan | 3.2.1.136 | Feraxan endoxylanase; | Endohydrolysis of 1,4-β-D- |
| | endo-1,4-β-xylanase | | Arabinoxylanase | xylosyl links in some |
| | | | | gluconoarabinoxylans |

“Ligninases” includes enzymes that can hydrolyze or break down the structure of lignin polymers. Enzymes that can break down lignin include lignin peroxidases, manganese peroxidases, laccases and feruloyl esterases, and other enzymes described in the art known to depolymerize or otherwise break lignin polymers. Also included are enzymes capable of hydrolyzing bonds formed between hemicellulosic sugars (notably arabinose) and lignin.

TABLE 3
Ligninases include, but are not limited to,
the following classes of enzymes

| Name Used | | | |
|---|---|---|---|
| in this | EC | | Reaction |
| application | Classification | Alternate Names | catalyzed |
| Lignin | 1.11.1 | none | Oxidative |
| peroxidase | | | degradation of |
| | | | lignin |
| Manganese | 1.11.1.13 | Mn-dependent | Oxidative |
| peroxidase | | peroxidase | degradation of |
| | | | lignin |
| Laccase | 1.10.3.2 | Urishiol oxidase | Oxidative |
| | | | degradation of |
| | | | lignin |
| Feruloyl | 3.1.1.73 | Ferulic acid esterase; | Hydrolyzes bonds |
| esterase | | Hydroxycinnamoyl | between |
| | | esterase; Cinnamoyl | arabinose and |
| | | ester hydrolase | lignin |

“Amylase” or “alpha glucosidase” includes enzymes that hydrolyze 1,4-alpha-glucosidic linkages in oligosaccharides and polysaccharides. Many amylases are characterized under the following EC listings:

TABLE 4
Amylases include, but are not limited to, the following classes of enzymes

| Name Used in | EC | | |
|---|---|---|---|
| this application | Classification | Alternate Names | Reaction catalyzed |
| α-amylase | 3.2.1.1 | 1,4-α-D-glucan | Hydrolysis of 1,4-α-glucosidic |
| | | glucanohydrolase; | linkages |
| | | Glycogenase | |
| β-amylase | 3.2.1.2 | 1,4-α-D-glucan | Hydrolysis of terminal 1,4-linked α- |
| | | maltohydrolase; | D-glucose residues |
| | | Saccharogen amylase | |
| | | Glycogenase | |
| Glucan 1,4-α- | 3.2.1.3 | Glucoamylase; 1,4-α- | Hydrolysis of terminal 1,4-linked α- |
| glucosidase | | D-glucan | D-glucose residues |
| | | glucohydrolase | |
| | | Amyloglucosidase; γ- | |
| | | amylase; Lysosomal | |
| | | α-glucosidase; Exo-1, | |
| | | 4-α-glucosidase | |
| α-glucosidase | 3.2.1.20 | Maltase; | Hydrolysis of terminal, non- |
| | | Glucoinvertase; | reducing 1,4-linked D-glucose |
| | | Glucosidosucrase; | |
| | | Maltase- | |
| | | glucoamylase; | |
| | | Lysosomal α- | |
| | | glucosidase; Acid | |
| | | maltase | |
| Glucan 1,4-α- | 3.2.1.60 | Exo- | Hydrolysis of 1,4-α-D-glucosidic |
| maltotetrahydrolase | | maltotetrahydrolase; | linkages |
| | | G4-amylase; | |
| | | Maltotetraose- | |
| | | forming amylase | |
| Isoamylase | 3.2.1.68 | Debranching enzyme | Hydrolysis of α-(1,6)-D-glucosidic |
| | | | Branco linkages in glycogen, |
| | | | amylopectin and their beta-limits |
| | | | dextrins |
| Glucan-1,4-α- | 3.2.1.98 | Exomaltohexaohydrolase; | Hydrolysis of 1,4-α-D-glucosidic |
| maltohexaosidase | | Maltohexaose- | linkages |
| | | producing amylase; | |
| | | G6-amylase | |
| Glucan-1,4-α- | 3.2.1.133 | Maltogenic α- | Hydrolysis of (1→4)-α-D-glucosidic |
| maltohydrolase | | amylase | linkages in polysaccharides |
| Cyclomaltodextrin | 2.4.1.19 | Cyclodextrin- | Degrades starch to cyclodextrins by |
| glucanotransferase | | glycosyltransferase; | formation of a 1,4-α-D-glucosidic |
| | | Bacillus macerans | bond |
| | | amylase; | |
| | | Cyclodextrin | |
| | | glucanotransferase | |
| Oligosaccharide 4- | 2.4.1.161 | Amylase III | Transfer the non-reducing terminal |
| α-D- | | | α-D-glucose residue from a 1,4-α-D- |
| glucosyltransferase | | | glucan to the 4-position of an α-D- |
| | | | glucan |

“Protease” includes enzymes that hydrolyze peptide bonds (peptidases), as well as enzymes that hydrolyze bonds between peptides and other moieties, such as sugars (glycopeptidases). Many proteases are characterized under EC 3.4, and are incorporated herein by reference. Some specific types of proteases include, cysteine proteases including pepsin, papain and serine proteases including chymotrypsins, carboxypeptidases and metalloendopeptidases. The SWISS-PROT Protein Knowledgebase (maintained by the Swiss Institute of Bioinformatics (SIB), Geneva, Switzerland and the European Bioinformatics Institute (EBI), Hinxton, United Kingdom) classifies proteases or peptidases into the following classes.

| | |
|---|---|
| Family | Representative enzyme |

Serine-type peptidases

| | |
|---|---|
| S1 | Chymotrypsin/trypsin |
| S2 | Alpha-Lytic endopeptidase |
| S2 | Glutamyl endopeptidase (V8) (Staphylococcus) |
| S2 | Protease Do (htrA) (Escherichia) |
| S3 | Togavirin |
| S5 | Lysyl endopeptidase |
| S6 | IgA-specific serine endopeptidase |
| S7 | Flavivirin |
| S29 | Hepatitis C virus NS3 endopeptidase |
| S30 | Tobacco etch virus 35 kDa endopeptidase |
| S31 | Cattle diarrhea virus p80 endopeptidase |
| S32 | Equine arteritis virus putative endopeptidase |
| S35 | Apple stem grooving virus serine endopeptidase |
| S43 | Porin D2 |
| S45 | Penicillin amidohydrolase |
| S8 | Subtilases |
| S8 | Subtilisin |
| S8 | Kexin |
| S8 | Tripeptidyl-peptidase II |
| S53 | Pseudomonapepsin |
| S9 | Prolyl oligopeptidase |
| S9 | Dipeptidyl-peptidase IV |
| S9 | Acylaminoacyl-peptidase |
| S10 | Carboxypeptidase C |
| S15 | Lactococcus X-Pro dipeptidyl-peptidase |
| S28 | Lysosomal Pro-X carboxypeptidase |
| S33 | Prolyl aminopeptidase |
| S11 | D-Ala-D-Ala peptidase family 1 (E. coli dacA) |
| S12 | D-Ala-D-Ala peptidase family 2 (Strept. R61) |
| S13 | D-Ala-D-Ala peptidase family 3 (E. coli dacB) |
| S24 | LexA repressor |
| S26 | Bacterial leader peptidase I |
| S27 | Eukaryote signal peptidase |
| S21 | Assemblin (Herpesviruses protease) |
| S14 | ClpP endopeptidase (Clp) |
| S49 | Endopeptidase IV (sppA) (E. coli) |
| S41 | Tail-specific protease (prc) (E. coli) |
| S51 | Dipeptidase E (E. coli) |
| S16 | Endopeptidase La (Lon) |
| S19 | Coccidiodes endopeptidase |
| S54 | Rhomboid |

Threonine-type peptidases

| | |
|---|---|
| T1 | Multicatalytic endopeptidase (Proteasome) |

Cysteine-type peptidases

| | |
|---|---|
| C1 | Papain |
| C2 | Calpain |
| C10 | Streptopain |
| C3 | Picornain |
| C4 | Potyviruses NI-a (49 kDa) endopeptidase |
| C5 | Adenovirus endopeptidase |
| C18 | Hepatitis C virus endopeptidase 2 |
| C24 | RHDV/FC protease P3C |
| C6 | Potyviruses helper-component (HC) proteinase |
| C7 | Chestnut blight virus p29 endopeptidase |
| C8 | Chestnut blight virus p48 endopeptidase |
| C9 | Togaviruses nsP2 endopeptidase |
| C11 | Clostripain |
| C12 | Ubiquitin C-terminal hydrolase family 1 |
| C13 | Hemoglobinase |
| C14 | Caspases (ICE) |
| C15 | Pyroglutamyl-peptidase I |
| C16 | Mouse hepatitis virus endopeptidase |
| C19 | Ubiquitin C-terminal hydrolase family 2 |
| C21 | Turnip yellow mosaic virus endopeptidase |
| C25 | Gingipain R |
| C26 | Gamma-glutamyl hydrolase |
| C37 | Southampton virus endopeptidase |
| C40 | Dipeptidyl-peptidase VI (Bacillus) |
| C48 | SUMO protease |
| C52 | CAAX prenyl protease 2 |

Aspartic-type peptidases

| | |
|---|---|
| A1 | Pepsin |
| A2 | Retropepsin |
| A3 | Cauliflower mosaic virus peptidase |
| A9 | Spumaretrovirus endopeptidase |
| A11 | Drosophila transposon copia endopeptidase |
| A6 | Nodaviruses endopeptidase |
| A8 | Bacterial leader peptidase II |
| A24 | Type IV-prepilin leader peptidase |
| A26 | Omptin |
| A4 | Scytalidopepsin |
| A5 | Thermopsin |

Metallopeptidases

| | |
|---|---|
| M1 | Membrane alanyl aminopeptidase |
| M2 | Peptidyl-dipeptidase A |
| M3 | Thimet oligopeptidase |
| M4 | Thermolysin |
| M5 | Mycolysin |
| M6 | Immune inhibitor A (Bacillus) |
| M7 | Streptomyces small neutral protease |
| M8 | Leishmanolysin |
| M9 | Microbial collagenase |
| M10 | Matrixin |
| M10 | Serralysin |
| M10 | Fragilysin |
| M11 | Autolysin (Chlamydomonas) |
| M12 | Astacin |
| M12 | Reprolysin |
| M13 | Neprilysin |
| M26 | IgA-specific metalloendopeptidase |
| M27 | Tentoxilysin |
| M30 | Staphylococcus neutral protease |
| M32 | Carboxypeptidase Taq |
| M34 | Anthrax lethal factor |
| M35 | Deuterolysin |
| M36 | Aspergillus elastinolytic metalloendopeptidase |
| M37 | Lysostaphin |
| M41 | Cell division protein ftsH (E. coli) |
| M46 | Pregnancy-associated plasma protein-A |
| M48 | CAAX prenyl protease |
| M49 | Dipeptidyl-peptidase III |

Others without HEXXH motifs

| | |
|---|---|
| M14 | Carboxypeptidase A |
| M14 | Carboxypeptidase H |
| M15 | Zinc D-Ala-D-Ala carboxypeptidase |
| M45 | Enterococcus D-Ala-D-Ala dipeptidase |
| M16 | Pitrilysin |
| M16 | Mitochondrial processing peptidase |
| M44 | Vaccinia virus-type metalloendopeptidase |
| M17 | Leucyl aminopeptidase |
| M24 | Methionyl aminopeptidase, type 1 |
| M24 | X-Pro dipeptidase |
| M24 | Methionyl aminopeptidase, type 2 |
| M18 | Yeast aminopeptidase I |
| M20 | Glutamate carboxypeptidase |
| M20 | Gly-X carboxypeptidase |
| M25 | X-His dipeptidase |
| M28 | Vibrio leucyl aminopeptidase |
| M28 | Aminopeptidase Y |
| M28 | Aminopeptidase iap (E. coli) |
| M40 | Sulfolobus carboxypeptidase |
| M42 | Glutamyl aminopeptidase (Lactococcus) |
| M38 | E. coli beta-aspartyl peptidase |
| M22 | O-Sialoglycoprotein endopeptidase |
| M52 | Hydrogenases maturation peptidase |
| M50 | SREBP site 2 protease |
| M50 | Sporulation factor IVB (B. subtilis) |
| M19 | Membrane dipeptidase |
| M23 | Beta-Lytic endopeptidase |
| M29 | Thermophilic aminopeptidase |

Peptidases of unknown catalytic mechanism

| | |
|---|---|
| U3 | Spore endopeptidase gpr (Bacillus) |
| U4 | Sporulation sigmaE factor processing peptidase (Bacillus) |
| U6 | Murein endopeptidase (mepA) (E. coli) |
| U8 | Bacteriophage murein endopeptidase |
| U9 | Prohead endopeptidase (phage T4) |
| U22 | Drosophila transposon 297 endopeptidase |
| U24 | Maize transposon bs1 endopeptidase |
| U26 | Enterococcus D-Ala-D-Ala carboxypeptidase |
| U29 | Encephalomyelitis virus endopeptidase 2A |
| U30 | Commelina yellow mottle virus proteinase |
| U31 | Human coronavirus protease |
| U32 | Porphyromonas collagenase |
| U33 | Rice tungro bacilliform virus endopeptidase |
| U34 | Lactococcal dipeptidase A |

“Lipase” includes enzymes that hydrolyze lipids, fatty acids, and acylglycerides, including phosphoglycerides, lipoproteins, diacylglycerols, and the like. In plants, lipids are used as structural components to limit water loss and pathogen infection. These lipids include waxes derived from fatty acids, as well as cutin and suberin. Many lipases are characterized under the following EC listings:

TABLE 5
Lipases include, but are not limited to, the following classes of enzymes

| Name Used in | EC | | |
|---|---|---|---|
| this application | Classification | Alternate Names | Reaction catalyzed |
| Triacylglycerol lipase | 3.1.1.3 | Lipase; Triglyceride | Triacylglycerol_H2O [symbol] |
| | | lipase; Tributyrase | diacylglycerol + a fatty acid anion |
| Phospholipase A2 | 3.1.1.4 | Phosphatidylcholine 2- | Phosphatidylcholine + H2O [symbol] 1- |
| | | acylhydrolase; Lecithinase | acylglycerophosphocholine + a fatty |
| | | A; Phosphatidase; | acid anion |
| | | Phosphatidolipase | |
| Lysophospholipase | 3.1.1.5 | Lecithinase B; | 2-lysophosphatidylcholine + H2O [symbol] |
| | | Lysolecithinase; | glycerophosphocholine + a fatty acid |
| | | Phospholipase B | anion |
| Acylglycerol lipase | 3.1.1.23 | Monoacylglycerol lipase | Hydrolyzes glycerol monoesters of |
| | | | long-chain fatty acids |
| Galactolipase | 3.1.1.26 | None | 1,2-diacyl-3-β-D-galactosyl-sn- |
| | | | glycerol + 2 H2O [symbol] 3-β-D- |
| | | | galactosyl-sn-glycerol + 2 fatty acid |
| | | | anion |
| Phospholipase A1 | 3.1.1.32 | None | Phosphatidylcholine + H2O [symbol] 2- |
| | | | acylglycerophosphocholine + a fatty |
| | | | acid anion |
| Dihydrocoumarin | 3.1.1.35 | None | Dihydrocoumarin + H2O [symbol] |
| lipase | | | melilotate |
| 2-acetyl-1- | 3.1.1.47 | 1-alkyl-2- | 2-acetyl-1-alkyl-sn-glycero-3- |
| alkylglycerophospho- | | acetylglycerophosphocholine | phosphocholine + H2O [symbol] 1-alkyl- |
| choline esterase | | esterase; Platelet- | sn-glycero-3-phosphocholine + |
| | | activating factor | acetate |
| | | acetylhydrolase; PAF | |
| | | acetylhydrolase; PAF 2- | |
| | | acylhydrolase; LDL- | |
| | | associated phospholipase | |
| | | A2; LDL-PLA(2) | |
| Phosphatidylinositol | 3.1.1.52 | Phosphatidylinositol | 1-phosphatidyl-1D-myoinositol + |
| deacylase | | phospholipase A2 | H2O [symbol] 1- |
| | | | acylglycerophosphoinositol + a fatty |
| | | | acid anion |
| Cutinase | 3.1.1.74 | None | Cutis + H2O [symbol] cutis monomers |
| Phospholipase C | 3.1.4.3 | Lipophosphodiesterase I; | A phosphatidylcholine + H2O [symbol] 1,2 |
| | | Lecithinase C; | diacylglycerol + choline phosphate |
| | | Clostridium welchii α- | |
| | | toxin; Clostridium | |
| | | oedematiens β- and γ- | |
| | | toxins | |
| Phospholipase D | 3.1.4.4 | Lipophosphodiesterase II; | A phosphatidylcholine + H2O [symbol] |
| | | Lecithinase D; Choline | choline + a phosphatidate |
| | | phosphatase | |
| 1- | 3.1.4.10 | Monophosphatidylinositol | 1-phosphatidyl-1D-myoinositol [symbol] |
| phosphatidylinositol | | phosphodiesterase; | 1D-mylinositol 1,2-cyclic phosphate + |
| phosphodiesterase | | Phosphatidylinositol | diacylglycerol |
| | | phospholipase C | |
| Alkylglycerophospho | 3.1.4.39 | Lysophospholipase D | 1-alkyl-sn-glycero-3- |
| ethanolamine | | | phosphoethanolamine + H2O [symbol] 1- |
| phosphodiesterase | | | alkyl-sn-glycerol 3-phosphate + |
| | | | ethanolamine |

“Glucuronidase” includes enzymes that catalyze the hydrolysis of beta-glucuroniside to yield an alcohol. Many glucoronidases are characterized under the following EC listings.

TABLE 6
Glucuronidases include, but are not limited, to the following classes of enzymes

| Name Used | | | |
|---|---|---|---|
| in this | EC | | |
| application | Classification | Alternate Names | Reaction catalyzed |
| β-glucuronidase | 3.2.1.31 | None | A beta-D-glucuronosidase + H2O [symbol] |
| | | | an alcohol + D-glucuronate |
| Hyalurono- | 3.2.1.36 | Hyaluronidase | Hydrolysis of 1,3-linkages between |
| glucuronidase | | | β-D-glucuronate and N-acetyl-D- |
| | | | glucosamine |
| Glucuronosyl- | 3.2.1.56 | None | 3-D-glucuronosyl-N (2)-6-disulfo-β- |
| disulfoglucos- | | | D-glucosamine + H2o [symbol] N (2)-6- |
| amine | | | disulfo-D-glucosamine + D- |
| glucuronidase | | | glucuronate |
| Glycyrrhizinate | 3.2.1.128 | None | Glycyrrhizinate + H2O [symbol] 1,2-β-D- |
| β-glucuronidase | | | glucuronosyl-D-glucuronate + |
| | | | glycyrrhetinate |
| α- | 3.2.1.139 | α-glucuronidase | An α-D-glucuronosidase + H2O [symbol] |
| glucosiduronase | | | an alcohol + D-glururonate |

### Enzyme Compositions

“At least one enzyme capable of hydrolyzing lignocellulose” or “at least one enzyme” is defined as any enzyme or mixture of enzymes that increases or enhances sugar release from biomass following a ‘treatment reaction’. This can include enzymes that when contacted with biomass in a reaction, increase the activity of subsequent enzymes. The treatment with an “enzyme” is referred to as an ‘enzymatic treatment’. Enzymes with relevant activities include, but are not limited to, cellulases, xylanases, ligninases, amylases, proteases, lipases and glucuronidases. Many of these enzymes are representatives of class EC 3.2.1, and thus other enzymes in this class may be useful in this invention. Two or more enzymes may be combined to yield an “enzyme mix” to hydrolyze lignocellulose during treatment. An enzyme mix may be composed of enzymes from (1) commercial suppliers; (2) cloned genes expressing enzymes; (3) complex broth (such as that resulting from growth of a microbial strain in media, wherein the strains secrete proteins and enzymes into the media), including broth from semi-solid or solid phase media, as well as broth containing the feedstock itself, (4) cell lysates of strains grown as in (3); and, (5) plant material expressing enzymes capable of hydrolyzing lignocellulose.

It is recognized that any combination of enzymes may be utilized. The enzymes may be used alone or in mixtures including, but not limited to, at least a cellulase; at least a xylanase; at least a ligninase; at least an amylase; at least a protease; at least a lipase; at least a glucuronidase; at least a cellulase and a xylanase; at least a cellulase and a ligninase; at least a cellulase and an amylase; at least a cellulase and a protease; at least a cellulase and a lipase; at least a cellulase and a glucuronidase; at least a xylanase and a ligninase; at least a xylanase and an amylase; at least a xylanase and a protease; at least a xylanase and a lipase; at least a xylanase and a glucuronidase; at least a ligninase and an amylase; at least a ligninase and a protease; at least a ligninase and a lipase; at least a ligninase and a glucuronidase; at least an amylase and a protease; at least an amylase and a lipase; at least an amylase and a glucuronidase; at least a protease and a lipase; at least a protease and a glucuronidase; at least a lipase and a glucuronidase; at least a cellulase, a xylanase and a ligninase; at least a xylanase, a ligninase and an amylase; at least a ligninase, an amylase and a protease; at least an amylase, a protease and a lipase; at least a protease, a lipase and a glucuronidase; at least a cellulase, a xylanase and an amylase; at least a cellulase, a xylanase and a protease; at least a cellulase, a xylanase and a lipase; at least a cellulase, a xylanase and a glucuronidase; at least a cellulase, a ligninase and an amylase; at least a cellulase, a ligninase and a protease; at least a cellulase, a ligninase and a lipase; at least a cellulase, a ligninase and a glucuronidase; at least a cellulase, an amylase and a protease; at least a cellulase, an amylase and a lipase; at least a cellulase, an amylase and a glucuronidase; at least a cellulase, a protease and a lipase; at least a cellulase, a protease and a glucuronidase; at least a cellulase, a lipase and a glucuronidase; at least a cellulase, a xylanase, a ligninase and an amylase; at least a xylanase, a ligninase, an amylase and a protease; at least a ligninase, an amylase, a protease and a lipase; at least an amylase, a protease, a lipase and a glucuronidase; at least a cellulase, a xylanase, a ligninase and a protease; at least a cellulase, a xylanase, a ligninase and a lipase; at least a cellulase, a xylanase, a ligninase and a glucuronidase; at least a cellulase, a xylanase, an amylase and a protease; at least a cellulase, a xylanase, an amylase and a lipase; at least a cellulase, a xylanase, an amylase and a glucuronidase; at least a cellulase, a xylanase, a protease and a lipase; at least a cellulase, a xylanase, a protease and a glucuronidase; at least a cellulase, a xylanase, a lipase and a glucuronidase; at least a cellulase, a ligninase, an amylase and a protease; at least a cellulase, a ligninase, an amylase and a lipase; at least a cellulase, a ligninase, an amylase and a glucuronidase; at least a cellulase, a ligninase, a protease and a lipase; at least a cellulase, a ligninase, a protease and a glucuronidase; at least a cellulase, a ligninase, a lipase and a glucuronidase; at least a cellulase, an amylase, a protease and a lipase; at least a cellulase, an amylase, a protease and a glucuronidase; at least a cellulase, an amylase, a lipase and a glucuronidase; at least a cellulase, a protease, a lipase and a glucuronidase; at least a cellulase, a xylanase, a ligninase, an amylase and a protease; at least a cellulase, a xylanase, a ligninase, an amylase and a lipase; at least a cellulase, a xylanase, a ligninase, an amylase and a glucuronidase; at least a cellulase, a xylanase, a ligninase, a protease and a lipase; at least a cellulase, a xylanase, a ligninase, a protease and a glucuronidase; at least a cellulase, a xylanase, a ligninase, a lipase and a glucuronidase; at least a cellulase, a xylanase, an amylase, a protease and a lipase; at least a cellulase, a xylanase, an amylase, a protease and a glucuronidase; at least a cellulase, a xylanase, an amylase, a lipase and a glucuronidase; at least a cellulase, a xylanase, a protease, a lipase and a glucuronidase; at least a cellulase, a ligninase, an amylase, a protease and a lipase; at least a cellulase, a ligninase, an amylase, a protease and a glucuronidase; at least a cellulase, a ligninase, an amylase, a lipase and a glucuronidase; at least a cellulase, a ligninase, a protease, a lipase and a glucuronidase; at least a cellulase, an amylase, a protease, a lipase and a glucuronidase; at least a xylanase, a ligninase, an amylase, a protease and a lipase; at least a xylanase, a ligninase, an amylase, a protease and a glucuronidase; at least a xylanase, a ligninase, an amylase, a lipase and a glucuronidase; at least a xylanase, a ligninase, a protease, a lipase and a glucuronidase; at least a xylanase, an amylase, a protease, a lipase and a glucuronidase; at least a ligninase, an amylase, a protease, a lipase and a glucuronidase; at least a cellulase, a xylanase, a ligninase, an amylase, a protease, and a lipase; at least a cellulase, a xylanase, a ligninase, an amylase, a protease and a glucuronidase; at least a cellulase, a xylanase, a ligninase, an amylase, a lipase and a glucuronidase; at least a cellulase, a xylanase, a ligninase, a protease, a lipase and a glucuronidase; at least a cellulase, a xylanase, an amylase, a protease, a lipase and a glucuronidase; at least a cellulase a ligninase, an amylase, a protease, a lipase, and a glucuronidase; at least a xylanase, a ligninase, an amylase, a protease, a lipase and a glucuronidase; at least a cellulase, a xylanase, a ligninase, an amylase, a protease, a lipase and a glucuronidase; and the like. It is understood that as described above, an auxiliary mix may be composed of a member of each of these enzyme classes, several members of one enzyme class (such as two or more xylanases), or any combination of members of these enzyme classes (such as a protease, an exocellulase, and an endoxylanase; or a ligninase, an exoxylanase, and a lipase).

The enzymes may be reacted with substrate or biomass simultaneously with the treatment or subsequent to the chemical treatment. Likewise if more than one enzyme is used the enzymes may be added simultaneously or sequentially. The enzymes may be added as a crude, semi-purified, or purified enzyme mixture. The temperature and pH of the substrate and enzyme combination may vary to increase the activity of the enzyme combinations. While the enzymes have been discussed as a mixture it is recognized that the enzymes may be added sequentially where the temperature, pH, and other conditions may be altered to increase the activity of each individual enzyme. Alternatively, an optimum pH and temperature can be determined for an enzyme mixture.

The enzymes are reacted with substrate under mild conditions. By “mild conditions” is intended conditions that do not include extreme heat or acid treatment, as is currently utilized for biomass conversion using bioreactors. For example, enzymes can be incubated at about 35° C. to about 65° C. in buffers of low to medium ionic strength, and neutral pH. By “medium ionic strength” is intended that the buffer has an ion concentration of about 200 millimolar (mM) or less for any single ion component. Incubation of enzyme combinations under these conditions results in release of substantial amounts of the sugar from the lignocellulose. By substantial amount or significant percentage is intended at least about 20%, about 30%, about 40%, about 50%, about 60%, about 70%, about 80%, about 85%, about 90%, about 95% and greater of available sugar.

### Enzyme Applications

The enzyme or enzymes used in the practice of the invention may be produced exogenously in microorganisms, yeasts, fungi, bacteria or plants, then isolated and added to the lignocellulosic feedstock. Alternatively, the organism producing the enzyme may be added into the feedstock. In this manner, plants that produce the enzymes may serve as the lignocellulosic feedstock and be added into lignocellulosic feedstock. The enzymes may also be produced in a fermentation organism producing a fermentation product, by simultaneous saccharification and fermentation.

Enzymes that degrade cellulose and hemicellulose are prevalent in nature, enabling organisms that produce them to degrade the more than 40 billion tons of cellulose biomass produced each year. Degradation of cellulose is a process that can involve as many as three distinct activities: 1) endoglucanases (EC 3.2.1.4), which cleave cellulose polymers internally; 2) cellobiohydrolases (EC 3.2.1.91), which attack cellulose polymers at non-reducing ends of the polymer; and, 3) beta-glucosidases (EC3.2.1.21), which cleave cellobiose dimers into glucose monomers and can cleave other small cellodextrins into glucose monomers. With these activities cellulose can be converted to glucose.

Likewise, hemicellulose can be converted to simple sugars and oligosaccharides by enzymes. In monocots, where heteroxylans are the principal constituent of hemicellulose, a combination of endo-1,4-beta-xylanase (EC 3.2.1.8) and beta-D-xylosidase (EC 3.2.1.37) may be used to break down hemicellulose to xylose. The mixed beta glucans are hydrolyzed by beta (1,3), (1,4) glucanases (EC 3.2.1.73).

Enzymes affecting biomass conversion are produced naturally in a wide range of organisms. Common sources are microorganisms including Trichoderma and Aspergillus species for cellulases and xylanases, and white rot fungi for ligninases. There are many organisms that have been noted to produce cellulases, cellobiohydrolases, glucosidases, xylanases, xylosidases, and ligninases. However, most of these enzymes have not been tested for their ability to degrade plant biomass, especially corn stover. Thus, the method of the invention can be used to test the use of enzymes in hydrolyzing corn stover and other lignocellulosic material.

As previously indicated, the enzymes or enzyme combinations can be expressed in microorganisms, yeasts, fungi or plants. Methods for the expression of the enzymes are known in the art. See, for example, Sambrook et al. (1989) Molecular Cloning: A Laboratory Manual (2d ed., Cold Spring Harbor Laboratory Press, Plainview, N.Y.); Ausubel et al., eds. (1995) Current Protocols in Molecular Biology (Greene Publishing and Wiley-Interscience, New York); U.S. Pat. Nos. 5,563,055; 4,945,050; 5,886,244; 5,736,369; 5,981,835; and others known in the art, all of which are herein incorporated by reference.

In one aspect of this invention the enzymes are produced in transgenic plants. Thus, the plant material comprising the lignocellulose may already comprise at least one enzyme capable of hydrolyzing lignocellulose. The lignocellulose may be incubated under conditions that allow the enzyme to hydrolyze lignocellulose prior to addition of the chemical. In addition, the lignocellulose may be subjected to processing, such as by modification of pH or washing, prior to addition of a chemical, or prior to any enzyme treatment. In this method the plants express the enzyme(s) that are required or contribute to biomass conversion to simple sugars or oligosaccharides. Such enzyme or enzyme combinations are sequestered or inactive to prevent hydrolysis of the plant during plant growth. In some cases where multiple enzymes display synergistic activity, one or more enzymes could be produced in the plant serving as the lignocellulosic feedstock and other enzymes produced in microorganism, yeast, fungi or another plant than the different enzyme sources mixed together with the feedstock to achieve the final synergistic mix of enzymes.

### Biomass Substrate Definitions

By “substrate”, “lignocellulose”, or “biomass” is intended materials containing cellulose, hemicellulose, lignin, protein, ash, and carbohydrates, such as starch and sugar. Component simple sugars include glucose, xylose, arabinose, mannose, and galactose. “Biomass” includes virgin biomass and/or non-virgin biomass such as agricultural biomass, commercial organics, construction and demolition debris, municipal solid waste, waste paper and yard waste. Common forms of biomass include trees, shrubs and grasses, wheat, wheat straw, sugar cane bagasse, corn, corn husks, corn kernel including fiber from kernels, products and by-products from milling of grains such as corn (including wet milling and dry milling) as well as municipal solid waste, waste paper and yard waste. “Blended biomass” is any mixture or blend of virgin and non-virgin biomass, preferably having about 5-95% by weight non-virgin biomass. “Agricultural biomass” includes branches, bushes, canes, corn and corn husks, energy crops, forests, fruits, flowers, grains, grasses, herbaceous crops, leaves, bark, needles, logs, roots, saplings, short rotation woody corps, shrubs, switch grasses, trees, vegetables, vines, and hard and soft woods (not including woods with deleterious materials). In addition, agricultural biomass includes organic waste materials generated from agricultural processes including farming and forestry activities, specifically including forestry wood waste. Agricultural biomass may be any of the aforestated singularly or in any combination of mixture thereof.

Biomass high in starch, sugar, or protein such as corn, grains, fruits and vegetables are usually consumed as food. Conversely, biomass high in cellulose, hemicellulose and lignin are not readily digestible and are primarily utilized for wood and paper products, fuel, or are typically disposed. Generally, the substrate is of high lignocellulose content, including corn stover, corn fiber, Distiller's dried grains, rice straw, hay, sugarcane bagasse, wheat, oats, barley malt and other agricultural biomass, switchgrass, forestry wastes, poplar wood chips, pine wood chips, sawdust, yard waste, and the like, including any combination of substrate.

Biomass may be used as collected from the field, or it may be processed, for example by milling, grinding, shredding, etc. Further, biomass may be treated by chemical or physical means prior to uses, for example by heating, drying, freezing, or by ensiling (storing for period of time at high moisture content). Such treatments include storage as bales, in open pits, as well as storage in reactors designed to result in modified properties such as microbial count or content, pH, water content, etc.

TABLE 7
Examples of materials typically referred to as biomass

| | | Residue from | |
|---|---|---|---|
| Non-Agricultural plant | Agricultural plant | Agricultural | Non-plant |
| material | material | processing | Material |
| Trees | Wheat straw | Corn Fiber | Refuse |
| Shrubs | Sugar cane bagasse | Residue from | Paper |
| | | agricultural | |
| | | crop processing | |
| Grasses | Rice Straw | | |
| Wood Chips | Switchgrass | | |
| Sawdust | Corn stover | | |
| Yard waste | Corn grain | | |
| Grass clippings | Corn fiber | | |
| Forestry wood waste | Vegetables | | |
| | Fruits | | |

By “liberate” or “hydrolysis” is intended the conversion of complex lignocellulosic substrates or biomass to simple sugars and oligosaccharides.

“Conversion” includes any biological, chemical and/or bio-chemical activity that produces ethanol or ethanol and byproducts from biomass and/or blended biomass. Such conversion includes hydrolysis, fermentation and simultaneous saccharification and fermentation (SSF) of such biomass and/or blended biomass. Preferably, conversion includes the use of fermentation materials and hydrolysis materials as defined herein.

“Corn stover” includes agricultural residue generated by harvest of corn plants. Stover is generated by harvest of corn grain from a field of corn, typically by a combine harvester. Corn stover includes corn stalks, husks, roots, corn grain, and miscellaneous material such as soil in varying proportions.

“Corn fiber” is a fraction of corn grain, typically resulting from wet milling or other corn grain processing. The corn fiber fraction contains the fiber portion of the harvested grain remaining after extraction of starch and oils. Corn fiber typically contains hemicellulose, cellulose, residual starch, protein and lignin.

“Ethanol” includes ethyl alcohol or mixtures of ethyl alcohol and water.

“Fermentation products” includes ethanol, lactic acid, citric acid, butanol and isopropanol as well as derivatives thereof.

“Distiller's dried grains” are the dried residue remaining after the starch fraction of corn has been removed for fermentation into ethanol. The material typically contains fiber, residual starch, protein and oils.

“Sugarcane bagasse” is a lignocellulosic product of sugarcane processing. The bagasse typically contains approximately 65% carbohydrates in the form of cellulose and hemicellulose.

“Malt” lignocellulose refers to barley malt utilized as a sugar source for brewing industries. The spent “malt” that is generated is high in cellulose, fiber and protein.

The following examples are offered by way of illustration and not by way of limitation.

### EXPERIMENTAL

### Example 1

### Glucose and Xylose Standard Curves

Standards for glucose, xylose, arabinose, galactose and mannose were prepared at concentrations ranging from 0%-0.12%. A modified dinitrosalicylic acid (DNS) method produced absorbance changes detected at 540 nm. A linear curve fit analysis for each sugar standard verifies that the DNS quantitation method is a precise detection method for each monomeric sugar (data not shown).

### Example 2

### Hydrogen Peroxide Treatment Followed by Cellulase Treatment Liberates Monomeric Sugars

Hydrogen peroxide (200 mM) was reacted with 2.0 g of stover in 10 mL water (adjusted to pH 5.0). A control stover sample was untreated. After 24 hours of incubation at 80° C., the reducing sugar content of each sample was determined by DNS assay (Example 1). Cellulase from T. longibrachiatum (25 mg) was then added to both samples and incubation was carried out for 24 hours at 65° C. The reducing sugars were determined by DNS assay. The results are shown in Table 8. Treatment with hydrogen peroxide resulted in greater sugar release after enzyme treatment than with enzyme alone.

TABLE 8
Reducing sugars solubilized from corn stover
Sugar Release following Treatment

| | |
|---|---|
| Stover only | 3.1% |
| Stover + H₂O₂ | 4.0% |
| Stover + Cellulase | 38.6% |
| Stover + H₂O₂ + Cellulase | 47.0% |

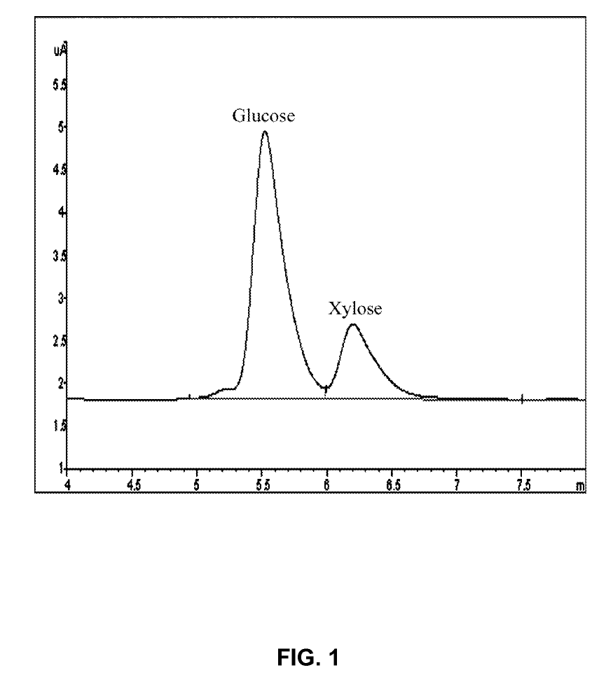

For further analysis by high performance liquid chromatography (HPLC), aliquots were removed, diluted 1:250 in water, and filtered using a 0.45 μm filter. The solubilized sugars were then separated at basic pH using an anion exchange HPLC column. Detection was carried out using an electrochemical detector in pulsed amperometric mode. External sugar standards (glucose, xylose) were used to identify glucose and xylose peaks. A chromatogram of sugars solubilized from stover following H₂O₂ and cellulase treatment is shown in FIG. 1.

### Example 3

### Hydrogen Peroxide Treatment Increases Enzymatic Hydrolysis of Corn Stover

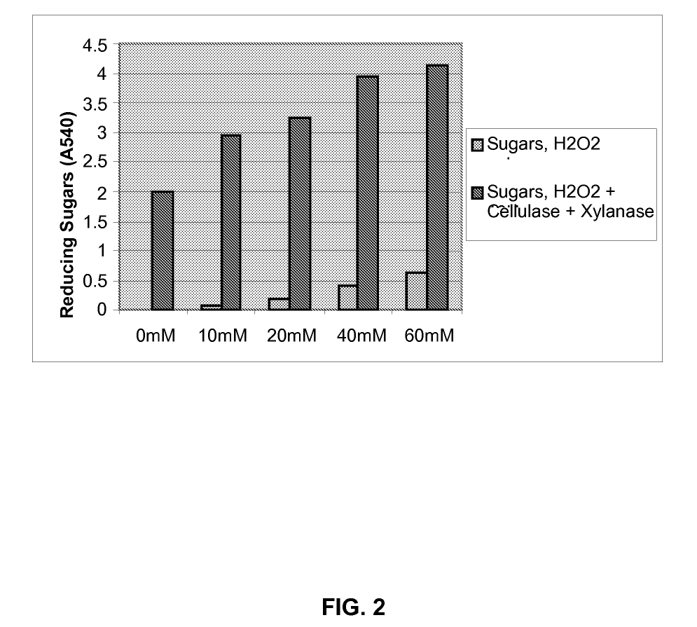

Hydrogen peroxide (0-60 mM final concentration) was reacted with 0.2 g stover in sodium acetate buffer (125 mM, pH 5.0) and incubated at 50° C. with shaking. After 24 hours, the reducing sugar content was determined by DNS assay. 10 units of cellulase from Trichoderma reesei and 10 units of xylanase from Trichoderma viride were then added and incubation was continued for 24 hours at 50° C. Additional aliquots were removed from each sample and reducing sugars quantified. The reducing sugar content following hydrogen peroxide treatment and enzymatic treatment is shown in FIG. 2. The amount of reducing sugars released was greater with increased concentration of hydrogen peroxide.

### Example 4

### Hydrogen Peroxide Breaks Down within 24 Hours of Treatment

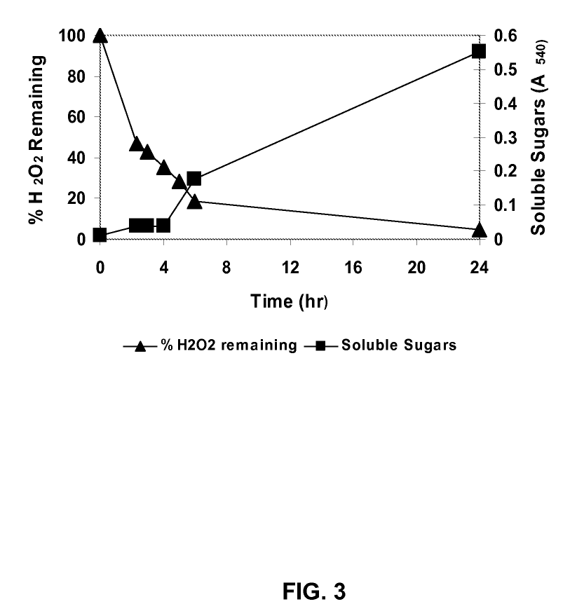

Hydrogen peroxide (0.13%) was reacted with 0.2 g stover in sodium acetate buffer (125 mM, pH 5.0) at 50° C. with shaking. Hydrogen peroxide was detected as follows (Kotterman (1986) App. Env. Microbiol. 62:880-885). Multiple aliquots (100 μL) from each sample were transferred to 96-well microtiter plates and mixed with 49 uL of 0.06% phenol red and 1 uL of 1.5 mg/mL horseradish peroxidase and incubated for 5 minutes. Samples were then mixed with 75 uL of 4N NaOH, quantitated at 610 nm, and compared to hydrogen peroxide standards. At timepoints from 0-24 hours, hydrogen peroxide and reducing sugars (DNS assay) were measured. These data are shown in FIG. 3. Control samples without stover did not change in their DNS assay and peroxide assay signals, respectively (data not shown). By 24 hours, the hydrogen peroxide concentration approached zero (FIG. 3). These results demonstrate that the treatment leaves a minimal chemical residue.

### Example 5

### Liberation of Sugars from Many Lignocellulose Materials

Lignocellulose material comprised of 1 gram of corn stover, corn fiber, Distiller's dried grains, Barley malt, or Sugarcane bagasse was mixed with hydrogen peroxide (100 mM) in 10 mL of water, and incubated for 24 hours at 80° C. Untreated reactions received no hydrogen peroxide. At the end of the incubation, the pH was adjusted by addition of 100 mM NaOAc buffer (pH 5.0), 25 mg of Trichoderma reesei cellulase was added, and the solution was incubated for 24 hours at 65° C. Untreated reactions received no cellulase. The reducing sugar content of the hydrolyzate was determined by DNS assay. The results of these experiments are shown in Table 9. These results show that the treatment is capable of releasing sugars from many lignocellulosic materials.

TABLE 9
Sugar release from lignocellulose materials
Percentage of Total Sugars Hydrolyzed

| Lignocellulose Material | Untreated | Treated |
|---|---|---|
| Corn Stover | 0.8% | 30.8% |
| Corn Fiber | 2.6% | 14.7% |
| Distiller's Dried Grains | 1.7% | 8.5% |
| Barley Malt | 0.9% | 16.7% |
| Sugarcane Bagasse | 1.1% | 10.6% |

### Example 6

### Production of Fermentable Materials from Corn Stover

Corn stover (2.0 g) was mixed with hydrogen peroxide (0.1%) in 10 mL of water. After 24 hours of incubation at 80° C., the pH was adjusted to 5.0 and 50 mg of cellulase from Trichoderma reesei was added and incubated for 24 hours at 65° C. The reducing sugar content of the hydrolyzate was then determined by DNS assay. Next, the hydrolyzate was adjusted to pH 7.0, filter-sterilized, and added to a carbon-free minimal growth media (M63) (Current Protocols in Molecular Biology, 2001) to produce a final sugar concentration of 5%. Control growth media was prepared by adding 5% glucose to media without sugar. Bacterial cells (Escherichia coli) were added to each medium, incubated with shaking at 37° C., and the growth was monitored through 48 hours by measuring the absorbance of each medium at 600 nm. The 48-hour timepoint for these data are shown in Table 10. Hydrolyzates of the method caused high levels of E. coli. growth. The results indicate that hydrolyzates from the method allow greater microbial growth than glucose. The hydrolyzates were not toxic to E. coli, even as undiluted hydrolyzates.

TABLE 10
Fermentative growth from corn stover hydrolyzate
Microbial Growth at 48 hours (A₆₀₀)

| | |
|---|---|
| No sugars | 0.0 |
| 5% Glucose | 1.2 |
| 5% Sugars from Stover | 2.1 |

### Example 7

### Hydrolyzates are Fermentable Materials That Enhance Microbial Growth

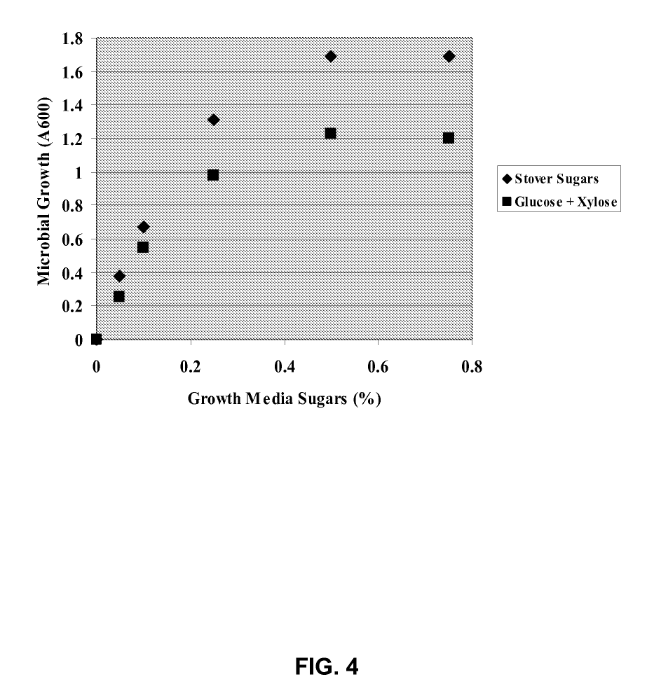

The hydrolyzate produced by hydrogen peroxide treatment and cellulase treatment (described in Example 6) was diluted into carbon-free minimal growth media (M63) to produce a final sugar concentration ranging from 0.0% to 1.0%. Control growth media were prepared with the same final sugar concentration of glucose and xylose (ratio of 63:37). Bacterial cells (Escherichia coli XL1 MRF′) were added to each medium, incubated with shaking at 37° C., and the growth was quantified at 48 hours by absorbance at 600 nm. Microbial growth was greater in the hydrolyzate media than in control media prepared with glucose and xylose (see FIG. 4).

### Example 8

### Detergent Treatment Increases Hydrolysis of Corn Stover by Hydrogen Peroxide Treatment Followed by Cellulase Treatment

Corn stover (2.0 g) was mixed with hydrogen peroxide (1%) in 10 mL of water. After 24 hours of incubation at 80° C., the pH was adjusted to 5.0. To this was added 50 mg of cellulase from Trichoderma reesei as well as Triton X-100 (2%, v/v). Separately, corn stover (2.0 g) was mixed with hydrogen peroxide (1%) in 10 mL of water, incubated for 24 hours at 80° C., and adjusted to pH 5.0. To this was added 50 mg of cellulase from Trichoderma reesei as well as Tween-20 (3%, v/v). Controls without detergent (cellulase only) were included in both experiments. Reactions were incubated for 96 hours at 40° C. The reducing sugar content was determined using the DNS assay. Results of this analysis show that both Tween-20 and Triton X-100 stimulate sugar release from corn stover. These data are summarized in Table 11.

TABLE 11
Effect of detergents on stover hydrolysis
Sugar Release following Treatment

| Detergent | Cellulase only | Cellulase + Detergent |
|---|---|---|
| Tween-20 | 39.2% | 44.7% |
| Triton X-100 | 30.7% | 38.1% |

### Example 9

### Oxidizing Agents Sterilize Lignocellulosic Materials

Corn stover (1 g) was suspended in 10 mL sterile water, and either autoclaved, or non-autoclaved. As expected, autoclaving killed essentially all microbes, resulting in less than 100 colony forming units per ml. In contrast, unautoclaved stover contained 20,000 colony forming units per mL. Unautoclaved samples were treated with 0.1% hydrogen peroxide at 50° C. for 24 hours. Serial dilutions were performed as known in the art and plated on nutrient broth plates. Plates were incubated at 30° C. for 24 hours, then colony forming units counted. Hydrogen peroxide treatment was found to reduce microbial content substantially compared to the untreated control (Table 12).

TABLE 12
Effect of hydrogen peroxide on microbial count of corn stover

| | Nonautoclaved (CFU/mL) | Nonautoclaved + H₂0₂ |
|---|---|---|
| Untreated, 0 hrs. | 28,500 | 18,000 |
| 24 hrs., 50° C. | 3,000 | 870 |

### Example 10

### Treatment of Biomass with Sodium Hypochlorite Increases Corn Stover Hydrolysis

Corn stover (0.2 g) was suspended in 9 mL of distilled water (pH 5.2) and 1 mL of sodium hypochlorite solution (10-13% available chlorine, Sigma). This pretreatment was carried out in a shaker-incubator at 80° C. at 300 rpm for 24 hours. Following pretreatment, the pH was adjusted to 5.2-5.4, and Spezyme CP (0.3 mL) (Genencor) was added to the samples followed by incubation at 40° C., 300 rpm for 24 hours. Supernatant aliquots were collected after 24 hours and the reducing sugar content was determined by DNS assay (λₘₐₓ=540 nm). All samples were run in duplicate. Sodium hypochlorite treatment produced significant hydrolysis of corn stover (Table 13). Treatment with 10% sodium hypochlorite and Spezyme resulted in greater hydrolysis of stover compared to treatment with Spezyme alone.

TABLE 13
Effects of sodium hypochlorite on stover hydrolysis
Sugar Release Following Treatment

| | Sodium Hypochlorite + | |
|---|---|---|
| | Spezyme | Spezyme |
| Sugar release | 71.9% | 32.8% |

Further quantification of sugars was performed by HPLC. HPLC chromatogram analysis of the treated material identifies the sugars produced following stover pretreatment using 10% NaOCl (24 hrs) followed by 0.3 mL of Spezyme (24 hrs). The sample was diluted by 1:50 prior to injection. A peak containing glucose, arabinose, galactose and mannose (6.3 minutes) was separated from a peak containing xylose (6.8 minutes). The percentage of available sugars solubilized was calculated by integration of each peak area (Table 14). Thus, treatment with sodium hypochlorite results in release of a high percentage of sugars from lignocellulose.

TABLE 14
Sugar yields following sodium hypochlorite and Spezyme treatment
Sugar Release Following Treatment

| | Glucose, Galactose, | | |
|---|---|---|---|
| | Arabinose, Mannose | Xylose | Total Sugars |
| % Sugars | 90% | 61% | 80% |
| Solubilized | | | |

### Example 11

### Significant Hydrolysis of Corn Stover is Obtained With Much Lower Concentrations of Cellulase

Stover samples pretreated with NaOCl were reacted with either 0.3 mL Spezyme or 0.03 mL Spezyme. Samples with 0.3 mL Spezyme produced 84% hydrolysis of total sugars, while samples with 0.03 mL Spezyme produced 79% hydrolysis. A control sample with no NaOCl and 0.3 mL Spezyme produced 42% hydrolysis (see Table 15). This experiment shows that pretreatment with a 10% solution of the NaOCl stock, followed by reaction with a cellulase (in this case Spezyme) produces significant hydrolysis of lignocellulose to sugar.

TABLE 15
Effect of Lower Enzyme on Hydrolysis Following Sodium Hypochlorite
Pretreatment
Sugar Release Following Treatment

| | | Sodium | Sodium |
|---|---|---|---|
| | 0.3 mL | Hypochlorite + | Hypochlorite + 0.03 mL |
| | Spezyme | 0.3 mL Spezyme | Spezyme |
| % Sugars | 42.6% | 84.6% | 76.0% |
| Solubilized | | | |

### Example 12

### Calcium Hypochlorite Treatment Increases Corn Stover Hydrolysis

Lignocellulose (corn stover, 0.2 g in final reaction of 10 mL) was contacted with calcium hypochlorite (1% available chlorine) at 80° C. for 24 hours. The pH was adjusted to pH 5.2, and 0.3 ml of Spezyme CP (Genencor) was added, and the reaction was incubated at 40° C. for 24 hours. Sugar release was measured by DNS assay. Treatment with calcium hypochlorite was found to increase sugar release beyond treatment with Spezyme alone (Table 16).

TABLE 16
Effects of calcium hypochlorite on stover hydrolysis
Sugar Release Following Treatment

| | Calcium Hypochlorite + | |
|---|---|---|
| | Spezyme | Spezyme |
| Sugar release | 71.4% | 26.4% |

### Example 13

### Urea Hydrogen Peroxide Increases Corn Stover Hydrolysis

Lignocellulose (corn stover, 0.2 g in final reaction of 10 mL) was contacted with 5% urea hydrogen peroxide (CAS# 124-43-6) at 80° C. for 24 hours. The stover was washed to dilute the chemical, the pH was adjusted to pH 5.2, 0.3 ml of Spezyme CP (Genencor) was added, and the reaction incubated at 40° C. for 48 hours. Sugar release was measured by DNS assay. Treatment with urea hydrogen peroxide was found to increase sugar release beyond treatment with Spezyme alone (Table 17).

TABLE 17
Effects of urea-hydrogen peroxide on stover hydrolysis
Sugar Release Following Treatment

| | Urea hydrogen peroxide + | |
|---|---|---|
| | Spezyme | Spezyme |
| Sugar release | 38.3% | 32.1% |

### Example 14

### N-methylmorpholine-N-oxide Increases Corn Stover Hydrolysis

Lignocellulose (corn stover, 0.2 g in final reaction of 10 mL) was contacted with 75% N-methylmorpholine-N-oxide (NMMO) (CAS #7529-22-8) at 80° C. for 24 hours.

The NMMO was then diluted, 0.3 ml of Spezyme CP (Genencor) was added, and the reaction incubated at 40° C. for 48 hours. Sugar release was measured by DNS assay.

Treatment with NMMO was found to release sugar above the amount released by treatment with Spezyme alone (Table 18).

TABLE 18
Effects of N-methylmorpholine-N-oxide on stover hydrolysis
Sugar Release Following Treatment

| | NMMO + Spezyme | Spezyme |
|---|---|---|
| Sugar release | 44.8% | 32.1% |

### Example 15

### Sodium Percarbonate Increases Corn Stover Hydrolysis

Lignocellulose (corn stover, 0.2 g in final reaction of 10 mL) was contacted with 2.5% sodium percarbonate (CAS# 15630-89-4) at 80° C. for 24 hours. The pH was adjusted to pH 5.2, 0.3 ml of Spezyme CP (Genencor) was added, and the reaction was incubated at 40° C. for 24 hours. Sugar release was measured by DNS assay. Treatment with sodium percarbonate was found to increase sugar release beyond treatment with Spezyme alone (Table 19).

TABLE 19
Effects of sodium percarbonate on stover hydrolysis
Sugar Release Following Treatment

| | Sodium Percarbonate + | |
|---|---|---|
| | Spezyme | Spezyme |
| Sugar release | 75.7% | 35.7% |

### Example 16

### Potassium Persulfate Increases Corn Stover Hydrolysis

Lignocellulose (corn stover, 0.2 g in final reaction of 10 mL) was contacted with 1% potassium persulfate (CAS#7727-21-1) at 80° C. for 24 hours. The pH was adjusted to pH 5.2, and 0.3 ml of Spezyme CP (Genencor) was added, and the reaction was incubated at 40° C. for 24 hours. Sugar release was measured by DNS assay. Treatment with potassium persulfate was found to increase sugar release beyond treatment with Spezyme alone (Table 20).

TABLE 20
Effects of potassium persulfate on stover hydrolysis
Sugar Release Following Treatment

| | Potassium Persulfate + | |
|---|---|---|
| | Spezyme | Spezyme |
| Sugar release | 44.8% | 35.9% |

### Example 17

### Peroxyacetic Acid Treatment Increases Corn Stover Hydrolysis

Lignocellulose (corn stover, 0.2 g in final reaction of 10 mL) was contacted with peroxyacetic acid (1% final concentration) at 80° C. for 24 hours. The pH was adjusted to pH 5.2, 0.3 ml of Spezyme CP (Genencor) was added, and the reaction was incubated at 40° C. for 96 hours. Sugar release was measured by DNS assay and HPLC. Treatment with peroxyacetic acid was found to increase sugar release beyond treatment with Spezyme alone (Table 21).

TABLE 21
Effects of peroxyacetic acid on stover hydrolysis
Sugar Release Following Treatment

| | Peroxyacetic Acid + | |
|---|---|---|
| | Spezyme | Spezyme |
| Sugar release | 69.9% | 38.5% |

### Example 18

### Potassium Superoxide Treatment Increases Corn Stover Hydrolysis

Lignocellulose (corn stover, 0.2 g in final reaction of 10 mL) was contacted with potassium superoxide (0.5% final concentration) at 80° C. for 24 hours. The pH was adjusted to pH 5.2, 0.3 ml of Spezyme CP (Genencor) was added, and the reaction was incubated at 40° C. for 96 hours. Sugar release was measured by DNS assay and HPLC. Treatment with potassium superoxide was found to increase sugar release beyond treatment with Spezyme alone (Table 22).

TABLE 22
Effects of potassium superoxide on stover hydrolysis
Sugar Release Following Treatment

| | Potassium Superoxide + | |
|---|---|---|
| | Spezyme | Spezyme |
| Sugar release | 89.1% | 38.5% |

### Example 19

### Sodium Carbonate Treatment Increases Corn Stover Hydrolysis

Lignocellulose (corn stover, 0.2 g in final reaction of 10 mL) was contacted with sodium carbonate (0.67% final concentration) to make a mixture with a pH of 10.0, which was incubated at 80° C. for 24 hours. The pH was adjusted to pH 5.2, 0.3 ml of Spezyme CP (Genencor) was added, and the reaction was incubated at 40° C. for 96 hours. Sugar release was measured by DNS assay and HPLC. Treatment with sodium carbonate was found to increase sugar release beyond treatment with Spezyme alone (Table 23).

TABLE 23
Effects of sodium carbonate on stover hydrolysis
Sugar Release Following Treatment

| | Sodium Carbonate + | |
|---|---|---|
| | Spezyme | Spezyme |
| Sugar release | 49.6% | 26.4% |

### Example 20

### Potassium Hydroxide Treatment Increases Corn Stover Hydrolysis

Lignocellulose (corn stover, 0.2 g in final reaction of 10 mL) was contacted with potassium hydroxide (75 mM final concentration) to make a mixture with a pH of 12.3, which was incubated at 80° C. for 24 hours. The pH was adjusted to pH 5.2, 0.3 ml of Spezyme CP (Genencor) was added, and the reaction was incubated at 40° C. for 96 hours. Sugar release was measured by DNS assay and HPLC. Treatment with potassium hydroxide was found to increase sugar release beyond treatment with Spezyme alone (Table 24).

TABLE 24
Effects of potassium hydroxide on stover hydrolysis
Sugar Release Following Treatment

| | Potassium Hydroxide + | |
|---|---|---|
| | Spezyme | Spezyme |
| Sugar release | 68.8% | 27.1% |

### Example 21

### Sodium Percarbonate Treatment Increases Hydrolysis of Corn Fiber Distiller's Dried Grains, Sugarcane Bagasse and Spent Barley Malt

Corn fiber, Distiller's dried grains, sugarcane bagasse and spent barley malt (0.2 g in final reaction of 10 mL) were each contacted with sodium percarbonate (1.0% final concentration) at 80° C. for 24 hours. The pH was adjusted to pH 5.2, 0.3 ml of Spezyme CP (Genencor) was added, and the reactions were incubated at 40° C. for 96 hours. Sugar release was measured by DNS assay and HPLC. Treatment with sodium percarbonate was found to increase sugar release beyond treatment with Spezyme alone (Table 25).

TABLE 25
Effects of sodium percarbonate treatment on various
biomass feedstocks
Sugar Release Following Treatment

| | Spezyme Percarbonate + | |
|---|---|---|
| | Spezyme | Spezyme only |
| Corn Fiber | 38.3% | 26.5% |
| Distiller's Dried Grains | 25.6% | 21.9% |
| Sugarcane Bagasse | 60.5% | 8.7% |
| Spent Barley Malt | 40.8% | 22.5% |

### Example 22

### Recycled Sodium Percarbonate Increases Corn Stover Hydrolysis

Corn stover (20 g in final reaction of 200 mL) was contacted with sodium percarbonate (5.0% final concentration) at 80° C. for 24 hours. The supernatant was removed and tested for the presence of sugars by DNS assay. The sugar concentration was less than 1%. This supernatant (10 mL) was contacted with fresh corn stover (0.2 g in final reaction of 10 mL) at 80° C. for 24 hours. In a separate reaction, freshly prepared sodium percarbonate (5.0% final concentration) was contacted with fresh corn stover (0.2 g in final reaction of 10 mL) at 80° C. for 24 hours. The pH of each sample was adjusted to pH 5.2, 0.3 ml of Spezyme CP (Genencor) was added, and the reactions were incubated at 40° C. for 96 hours. Sugar release was measured by DNS assay. Treatment with the recycled sodium percarbonate solution was found to increase sugar release beyond treatment with Spezyme alone (Table 26).

TABLE 26
Recycled sodium percarbonate increases hydrolysis of corn stover
Sugar Release Following Treatment

| | | | 5% Recycled |
|---|---|---|---|
| | | 5% Fresh Sodium | Sodium |
| | | Percarbonate + | Percarbonate + |
| | Spezyme | Spezyme | Spezyme |
| % Sugars | 31.2% | 79.3% | 83.5% |
| Solubilized | | | |

### Example 23

### Multiple Treatments Release Additional Sugar from Lignocellulose

Lignocellulose (corn stover, 0.2 g in final reaction of 10 mL) was contacted with 0.2% hydrogen peroxide at 80° C. for 24 hours. The pH was adjusted to pH 5.2, and 0.3 ml of Spezyme CP (Genencor) was added, and the reaction was incubated at 40° C. for 72 hours. Sugar release was measured by DNS assay, and each sample was then rinsed to remove soluble sugars. Next, hydrogen peroxide (0.2%), urea hydrogen peroxide (5%), sodium hypochlorite (1% available chlorine), calcium hypochlorite (1% available chlorine), or NMMO (75%) were added to individual samples, and incubated at 80° C. for 24 hours. Controls without chemical were also prepared. Following dilution of the chemical (NMMO) or simple pH adjustment to pH 5.2 (hydrogen peroxide, sodium hypochlorite, calcium hypochlorite, urea hydrogen peroxide, no chemical), 0.3 mL of Spezyme was added, and the reaction incubated at 40° C. for 72 hours. The second Spezyme treatment was found to increase sugar release when a second chemical treatment preceded it (Table 27).

TABLE 27
Effects of multiple treatments on stover hydrolysis

| Chemical Added | | Sugar Release | |
|---|---|---|---|
| Preceding | | Following 1^{st} | Following |
| 1^{st} Spezyme | Preceding | Spezyme | 2^{nd} Spezyme |
| Treatment | 2^{nd} Spezyme Treatment | Treatment | Treatment |
| Hydrogen | None | 37.3% | 5.3% |
| Peroxide | | | |
| Hydrogen | Hydrogen Peroxide + | 37.3% | 10.7% |
| Peroxide | Spezyme | | |
| Hydrogen | Sodium Hypochlorite + | 37.0% | 44.7% |
| Peroxide | Spezyme | | |
| Hydrogen | Calcium Hypochlorite + | 37.8% | 54.2% |
| Peroxide | Spezyme | | |
| Hydrogen | Urea Hydrogen Peroxide + | 36.3% | 24.3% |
| Peroxide | Spezyme | | |
| Hydrogen | NMMO + Spezyme | 37.1% | 22.2% |
| Peroxide | | | |

### Example 24

### Hydrogen Peroxide Treatment Generates Lignocellulose and Hydrolyzates that Support Lactic Acid Production

Lignocellulose (corn stover) was contacted with 0.2% hydrogen peroxide at 80° C. for 24 hours. The pH was adjusted to pH 5.2, and 0.3 ml of Spezyme CP (Genencor) was added, and the reaction was incubated at 40° C. for 72 hours. The residual solids were separated from the hydrolyzate, washed, suspended in water, and 0.01 g of a commercially available silage inoculant known to contain lactic acid-producing bacteria (Biotal Silage II Inoculant, Biotal Inc.) was added. Fermentation was carried out for 24 hours at 37° C., and microbial growth was confirmed microscopically. Similarly, the hydrolyzate generated following each treatment was adjusted to pH 7.0, filter-sterilized, mixed with a minimal salts medium lacking carbon (Enriched Minimal Media (EMM) EMM contains Solution A (In 900 mls: 2 g NaNO₃, 1.0 ml 0.8 M MgSO₄, 1.0 ml 0.1 M CaCl₂, 1.0 ml Trace Elements Solution (In 100 ml of 1000× solution: 0.1 g FeSO₄.7H₂O, 0.5 mg CuSO₄.5H₂O, 1.0 mg H₃BO₃, 1.0 mg MnSO₄.5H₂O, 7.0 mg ZnSO₄.7H₂O, 1.0 mg MoO₃, 4.0 g KCl)) and Solution B (In 100 mls: 0.21 g Na₂HPO₄, 0.09 g NaH₂PO₄, pH 7.0), and inoculated with a Biotal inoculant seed culture that was grown in MRS broth to A₆₀₀=0.5, washed twice, and diluted 1:1000. After incubation, fermentation liquid from both fermentations (stover residual solids and stover hydrolyzates) were assayed for production of NADH (340 nm) following enzymatic conversion of lactic acid to produce pyruvate (Diffchamb) (Table 28). Therefore, both the corn stover residual solids and the hydrolyzate produced are capable of supporting growth of lactic acid bacteria, and of supporting lactic acid production.

TABLE 28
Lactic acid production after hydrogen peroxide treatment of corn stover
Lactic Acid Production (340 nm)

| | |
|---|---|
| Biotal + Stover Hydrolyzate | 0.323 |
| Biotal + Stover Residual | 0.669 |
| Solids | |
| Stover Hydrolyzate only | 0.000 |
| Stover Residual Solids only | −0.009 |
| Biotal Inoculant only | −0.002 |

### Example 25

### Hydrogen Peroxide Treatment of Corn Fiber Generates Hydrolyzates and Residual Solids that Support Lactic Acid Production

Lignocellulose (corn fiber) was contacted with 0.2% hydrogen peroxide at 80° C. for 24 hours. The pH was adjusted to pH 5.2, 0.3 ml of Spezyme CP (Genencor) was added, and the reaction was incubated at 40° C. for 48 hours. The residual solids (0.2 g) were separated from the hydrolyzate, washed, suspended in water, and 0.01 g of a commercially available silage inoculant known to contain lactic acid-producing bacteria (Biotal Silage II Inoculant, Biotal Inc.) was added. Fermentation was carried out for 24 hours at 37° C., and microbial growth was confirmed microscopically. The hydrolyzate generated following treatment were adjusted to pH 7.0, filter-sterilized, mixed with a minimal salts medium lacking carbon (EMM), and also inoculated with a Biotal inoculant seed culture that was grown in MRS broth to A₆₀₀=0.5, washed, and diluted 1:1000. These fermentations were carried out for 64 hours at 37° C. After incubation, fermentation liquid from both fermentations (stover residual solids and stover hydrolyzate) were assayed for production of NADH (340 nm) following enzymatic conversion of lactic acid to produce pyruvate (Diffchamb) (Table 29). Therefore, both the corn fiber residual solids and the hydrolyzate produced are capable of supporting growth of lactic acid bacteria, and are capable of supporting lactic acid production.

TABLE 29
Lactic acid production after hydrogen peroxide treatment of corn fiber
Lactic Acid Production (340 nm)

| | |
|---|---|
| Biotal + Corn Fiber | 0.587 |
| Hydrolyzate | |
| Biotal + Corn Fiber | 0.026 |
| Residual Solids | |
| No Hydrolyzate | −0.002 |

### Example 26

### Treatment with Oxidizing Agents Generates Hydrolyzates that Support Lactic Acid Production

Corn stover was treated with hydrogen peroxide (0.2%) for 24 hours at 80° C., adjusted to pH 5.2, and treated with 0.3 mL Spezyme for 144 hours at 40° C. The stover was then rinsed, sterilized and 1 gram was contacted with urea hydrogen peroxide (5%) at 80° C. for 24 hours. Following pH adjustment to pH 5.2, 0.3 mL of Spezyme was added for 48 hours at 40° C. Similarly, 1.5 g of fresh corn stover was contacted with sodium hypochlorite (1% available chlorine) for 24 hours at 80° C., adjusted to pH 5.2, and then 0.3 mL of Spezyme CP was added for 48 hours at 40° C. Both hydrolyzates were then adjusted to pH 7.0, filter sterilized, and mixed with a minimal salts medium lacking carbon (EMM) at 0.2% total sugars concentration. A seed culture in MRS broth (Difco) containing a mixed lactic acid inoculant preparation (Biotal Silage Inoculant II, Biotal Inc.) was grown to A₆₀₀=0.5, washed twice, diluted 1:1000, added to each medium and incubated for 64 hours at 37° C. After incubation, fermentation liquid from both fermentations (urea hydrogen peroxide treated, sodium hypochlorite treated) were assayed for production of NADH (340 nm) following enzymatic conversion of lactic acid to produce pyruvate (Diffchamb) (Table 30). Therefore, hydrolyzates resulting from treatment of lignocellulosic materials with oxidizing agents can be used by lactic acid-producing bacteria and can be used to produce lactic acid.

TABLE 30
Lactic acid production after treatment with oxidizing agents
Lactic Acid Production from Biotal Inoculant (340 nm)

| | |
|---|---|
| Stover Hydrolyzate | 0.193 |
| following Urea Hydrogen | |
| Peroxide Treatment | |
| Stover Hydrolyzate | 0.133 |
| following Sodium | |
| Hypochlorite Treatment | |
| No Hydrolyzate | 0.003 |

### Example 27

### Hydrolyzates from Chemical Treatments Support Microbial Growth

Several corn stover hydrolyzates were prepared using chemical treatments in reaction volumes of 10 mL:

### Spezyme Only:

1.5 g corn stover was treated with 0.3 mL Spezyme CP (Genencor) for 48 hours, 40° C., at pH 5.2.

### Hydrogen Peroxide:

1.5 g corn stover was treated with 0.2% hydrogen peroxide (80° C., 24 hours), adjusted to pH 5.2, and then treated with 0.3 mL Spezyme CP (40° C., 48 hours).

### Sodium Hypochlorite:

1.5 g corn stover was treated with sodium hypochlorite (1% available chlorine) (80° C., 24 hours), adjusted to pH 5.2, and then treated with 0.3 mL Spezyme CP (40° C., 48 hours).

### Sodium Hypochlorite, Diluted:

1.5 g corn stover was treated with sodium hypochlorite (1% available chlorine) (80° C., 24 hours), washed to dilute the chemical, adjusted to pH 5.2, and then treated with 0.3 mL Spezyme CP (40° C., 48 hours).

### Urea Hydrogen Peroxide:

1.5 g corn stover was treated with 0.2% hydrogen peroxide (80° C., 24 hours), adjusted to pH 5.2, and then treated with 0.3 mL Spezyme CP (40° C., 48 hours).
The material was then treated with 10% urea hydrogen peroxide (80° C., 24 hours), adjusted to pH 5.2, and then treated with 0.3 mL Spezyme CP (40° C., 48 hours).

### Sodium Percarbonate:

0.2 g corn stover was treated with 2.5% sodium percarbonate (80° C., 24 hours), adjusted to pH 5.2, and then treated with 0.3 mL Spezyme CP (40° C., 48 hours).

### Potassium Persulfate:

0.2 g corn stover was treated with 1.0% potassium persulfate (80° C., 24 hours), adjusted to pH 5.2, and then treated with 0.3 mL Spezyme CP (40° C., 48 hours).

### Nitric Acid:

0.2 g corn stover was treated with 1.0% nitric acid (80° C., 24 hours), adjusted to pH 5.2, and then treated with 0.3 mL Spezyme CP (40° C., 48 hours).

Additionally, corn fiber hydrolyzate was prepared using hydrogen peroxide: 2 g corn fiber was treated with 0.2% hydrogen peroxide (80° C., 24 hours), adjusted to pH 5.2, and then treated with 0.3 mL Spezyme CP (40° C., 48 hours).

Following Spezyme treatment, each hydrolyzate was adjusted to pH 7.0, filter sterilized, and then added to a minimal salts medium lacking carbon (EMM) at a final sugars concentration of 0.2%. A negative control medium without sugars was also prepared. Each hydrolyzate was inoculated with a representative bacterial strain (ATX 3661) and incubated for 14 hours (no sugars, sodium hypochlorite diluted, urea hydrogen peroxide, sodium percarbonate, potassium persulfate, hydrogen peroxide) or 40 hours (hydrogen peroxide) or 48 hours (Spezyme only, sodium hypochlorite) at 37° C. Growth from each culture was assessed by absorbance at 600 nm (Table 31). Control cultures without sugars in each experiment yielded an absorbance (600 nm) lower than 0.005.

Therefore, hydrolyzates resulting from treatment of lignocellulosic material with various chemicals support microbial growth.

TABLE 31
Microbial growth following mild chemical treatment
Fermentative Growth, 14 hours, A₆₀₀

| Lignocellulosic | | |
|---|---|---|
| Substrate | Chemical | Growth (600 nm) |
| None | — | <0.005 |
| Corn Stover | None (Spezyme only) | 1.064 |
| Corn Stover | Hydrogen peroxide | 1.511 |
| Corn Stover | Sodium hypochlorite | 0.428 |
| Corn Stover | Sodium hypochlorite, diluted | 0.131 |
| Corn Stover | Urea hydrogen peroxide | 0.877 |
| Corn Stover | Sodium percarbonate | 0.692 |
| Corn Stover | Potassium persulfate | 0.641 |
| Corn Fiber | Hydrogen peroxide | 0.585 |

### Example 28

### Corn Stover Hydrolyzates Provide Components for Microbial Growth

ATX3661 is a Bacillus strain that will not grow in minimal media (EMM) when supplemented with glucose, or with glucose/xylose mixtures. Thus, ATX3661 requires additional nutrients other that glucose and xylose for growth in this media.

Lignocellulose (corn stover) was contacted with hydrogen peroxide (0.2%) or sodium hypochlorite (1% available chlorine) and incubated at 80° C. for 24 hours. The pH was adjusted to pH 5.2, 0.3 ml of Spezyme CP (Genencor) was added, and the reaction was incubated at 40° C. for 144 hours (sodium hypochlorite) or 48 hours (hydrogen peroxide). Corn stover samples without chemical treatment were included, and treated with Spezyme for 24 hours at 40° C. The hydrolyzates generated following Spezyme treatment were adjusted to pH 7.0, filter-sterilized, and mixed with a minimal salts medium lacking carbon (EMM) at a total sugar concentration of 0.20% (hydrogen peroxide) or 0.15% (sodium hypochlorite, Spezyme only). Control media was prepared in which glucose (0.095%) and xylose (0.055%) were added in place of the hydrolyzates (“Glucose/Xylose”), or hydrolyzate was omitted (“No Sugars”). Next, each media was inoculated with a representative bacterial strain (ATX 3661), incubated for 48 hours (sodium hypochlorite, Spezyme only, No Sugars, Glucose/Xylose) or 40 hours (hydrogen peroxide) at 37° C. Growth from each culture was detected by absorbance at 600 nm (Table 32). As expected, ATX3661 did not grow in EMM supplemented with Glucose and xylose. Surprisingly, ATX3661 did show growth when supplemented with hydrolyzates. Therefore, hydrolyzates supports microbial growth of strains that pure sugar does not.

TABLE 32
Effect of Hydrolyzate Components on Microbial Growth
Fermentative Growth, 14 hours, A₆₀₀

| Hydrolyzate or Sugars | Growth |
|---|---|
| No Sugars | −0.003 |
| Spezyme only | 1.064 |
| Hydrogen Peroxide + Spezyme | 1.511 |
| Sodium Hypochlorite + Spezyme | 0.428 |
| Glucose/Xylose + Spezyme | −0.001 |

### Example 29

### Hydrogen Peroxide Treatment and Sodium Percarbonate Treatment Increase Hydrolysis of Paper

Multipurpose copy paper (0.2 g, Quill, #7-20222) was shredded (average particle size=5 mm) and contacted with hydrogen peroxide (0.3% final concentration) or sodium percarbonate (1.0% final concentration) in a volume of 10 mL at 80° C. for 24 hours. The pH was adjusted to pH 5.2, 0.3 ml of Spezyme CP (Genencor) was added, and the reaction was incubated at 40° C. for 96 hours. Sugar release was measured by DNS assay. Treatment with hydrogen peroxide was found to increase sugar release beyond treatment with Spezyme alone (Table 33).

TABLE 33
Effect of hydrogen peroxide and sodium percarbonate on
paper hydrolysis
Sugar Release From Paper

| | Hydrogen Peroxide + | Sodium Percarbonate + |
|---|---|---|
| Spezyme only | Spezyme | Spezyme |
| 62.1% | 77.4% | 76.1% |

### Example 30

### Sodium Percarbonate and Potassium Superoxide Solubilize Corn Stover Proteins During Pretreatment

Lignocellulose (corn stover, 0.2 g in final reaction of 10 mL) was contacted with sodium percarbonate (1.0% final concentration) or potassium superoxide (0.5% final concentration) at 80° C. for 24 hours. The pH was adjusted to pH 5.2, and the supernatants tested for the presence of soluble protein (Bio-Rad Protein Assay). Bovine serum albumin (BSA) was used to generate a standard curve for quantitation. Treatment with sodium percarbonate or potassium superoxide was found to solubilize proteins from corn stover (Table 34).

TABLE 34
Solubilized protein is generated following pretreatment with sodium
percarbonate or potassium superoxide.
Protein Release Following Pretreatment

| | No | 1% Sodium | 0.5% Potassium |
|---|---|---|---|
| | pretreatment | Percarbonate | Superoxide |
| Protein Solubilized | 13 | 206 | 301 |
| (micrograms/milliliter) | | | |

### Example 31

### Sodium Hypochlorite Treatment at pH 5 Increases Corn Stover Hydrolysis

Lignocellulose (corn stover, 0.2 g in final reaction of 10 mL) was contacted with sodium hypochlorite (1% available chlorine, final concentration) at 80° C. for 24 hours. The pH was held constant by buffering with 200 mM sodium acetate buffer, pH 5, and a buffer-only negative control was also treated. 0.03 mL of Spezyme CP (Genencor) was added, and the reaction incubated at 40° C. for 96 hours. Sugar release was measured by DNS assay. Sodium hypochlorite treatment at pH 5 was found to increase sugar release beyond treatment with Spezyme alone (Table 35).

TABLE 35
Sodium hypochlorite buffered to pH 5.0 increases corn
stover hydrolysis
Sugar Release Following Treatment

| | | Sodium | |
|---|---|---|---|
| | | Hypochlorite | Buffer only |
| | | (buffered with | pretreatment |
| | Spezyme only, | Sodium Acetate, pH | (Sodium Acetate, |
| | unbuffered | 5.0) + Spezyme | pH 5.0) + Spezyme |
| % Sugars | 28.2% | 69.0% | 25.1% |
| Solubilized | | | |

### Example 32

### Peroxyacetic Acid Treatment Increases Corn Stover Hydrolysis in the Presence of Acetic Acid and Sulfuric Acid

Lignocellulose (corn stover, 0.2 g in final reaction of 10 mL) was contacted with peroxyacetic acid (Sigma Chemical, 2.0% final concentration). Since this reagent contains acetic acid and sulfuric acid as well, a mixture of acetic acid (2.6% final concentration) and sulfuric acid (0.06% final concentration) was used as a control. Reactions were incubated at 80° C. for 24 hours. Then, 0.03 mL of Spezyme CP (Genencor) was added to both reactions and they were incubated at 40° C. for 24 hours. Sugar release was measured by DNS assay. Peroxyacetic acid was found to liberate sugar from stover (Table 36).

TABLE 36
Peroxyacetic acid pretreatment increases corn stover hydrolysis
Sugar Release Following Treatment

| | | | Acetic Acid/Sulfuric |
|---|---|---|---|
| | | Acetic Acid/Sulfuric | Acid/Peroxyacetic |
| | | Acid Pretreatment + | Acid Pretreatment + |
| | Spezyme only | Spezyme | Spezyme |
| % Sugars | 19.4% | 15.3% | 49.0% |
| Solubilized | | | |

### Example 33

### Sodium Percarbonate, Sodium Hypochlorite and Peroxyacetic Acid Pretreatments Allow Hydrolysis with Low Enzyme Loads

Lignocellulose (corn stover, 0.2 g in final reaction of 10 mL) was contacted with sodium percarbonate (1.0% final concentration) or sodium hypochlorite (1% free chlorine, final concentration) or peroxyacetic acid (2.0% final concentration) at 80° C. for 24 hours. 0.03 mL or 0.012 mL or 0.006 mL of Spezyme CP (Genencor) was added, and the reaction was incubated at 40° C. for 120 hours. Sugar release was measured by DNS assay. Pretreatment with sodium percarbonate, sodium hypochlorite, or peroxyacetic acid allowed low enzyme concentrations to be used (Table 37).

TABLE 37
Sodium percarbonate, sodium hypochlorite and peroxyacetic
acid pretreatments allow hydrolysis with low enzyme loads
Sugar Release Following Treatment

| | 0.03 mL | | |
|---|---|---|---|
| | Spezyme | 0.012 mL Spezyme | 0.006 mL Spezyme |
| No Pretreatment | 19.8% | 24.2% | 27.0% |
| 1% Sodium | 45.8% | 55.0% | 67.3% |
| Percarbonate | | | |
| 1% Sodium | 62.0% | 71.4% | 76.0% |
| Hypochlorite | | | |
| 2% Peroxyacetic | 56.8% | 64.0% | 66.4% |
| Acid | | | |

### CONCLUSIONS

The results shown above demonstrate that the methods of the invention provide many advantages useful for lignocellulose degradation. These advantages include (1) the ability to use reactors with simple designs, (2) and the ability to reduce the amount of enzyme used in such processes, (3) the ability to produce and use a concentrated sugar solution, (4) the ability to directly use the treated product for fermentation without the need for further processing, as no toxic products are formed. These advantages also lead to economic benefits.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for hydrolyzing lignocellulose, comprising contacting said lignocellulose with at least one chemical under moderate conditions to generate a treated lignocellulose, and contacting said treated lignocellulose with at least one enzyme capable of hydrolyzing lignocellulose, wherein said chemical is selected from the group consisting of detergents, organic solvents, and combinations thereof, wherein said moderate conditions comprise:
a) a temperature from 10° C. to 90° C.;
b) a pressure less than 2 atm; and,
c) a pH between pH 4.0 and pH 10.0.

2. The method of claim 1, wherein said chemical comprises an organic solvent.

3. The method of claim 1, wherein said chemical comprises a detergent.

4. The method of claim 1, further comprising subjecting said lignocellulose to at least one physical treatment selected from the group consisting of grinding, milling, boiling, freezing, and vacuum filtration.

5. The method of claim 1, wherein said moderate conditions comprise a temperature of about 80° C.

6. The method of claim 1, wherein said moderate conditions comprise a pH of about pH 5.0.

7. The method of claim 1, wherein said contact occurs for about 24 hours.

8. The method of claim 1, wherein said enzyme comprises at least one enzyme selected from the group consisting of cellulase, xylanase, ligninase, amylase, protease, lipase, and glucuronidase.

9. The method of claim 1, wherein said temperature is adjusted to be optimal for said enzyme prior to enzyme addition.

10. The method of claim 1, wherein said pH is adjusted to be optimal for said enzyme prior to enzyme addition.

11. The method of claim 1, wherein said chemical is removed prior to addition of said enzyme.

12. The method of claim 1, further comprising removal of said chemical from said treated lignocellulose prior to additional treatment to obtain a recycled chemical.

13. The method of claim 1, wherein contacting said lignocellulose with at least one chemical occurs simultaneously with contacting said lignocellulose with at least one enzyme capable of hydrolyzing lignocellulose.

14. The method of claim 1, further comprising the addition of at least one fermenting organism, wherein said method results in the production of at least one fermentation-based product.

15. The method of claim 14, wherein said product is selected from the group consisting of lactic acid, a fuel, an organic acid, an industrial enzyme, a pharmaceutical, and an amino acid.

16. A method for pretreating a lignocellulosic material comprising contacting said material with at least one chemical under moderate conditions to generate a treated lignocellulose, wherein said chemical is selected from the group consisting of detergents, organic solvents, and a combination thereof, wherein said moderate conditions comprise:
a) a temperature from 10° C. to 90° C.;
b) a pressure less than 2 atm; and,
c) a pH between pH 4.0 and pH 10.0.

17. A method for liberating a substance from plant material, comprising contacting said plant material with at least one chemical under the following conditions:
a) a temperature from 10° C. to 90° C.;
b) a pressure less than 2 atm; and,
c) a pH between pH 4.0 and pH 10.0,
to generate a treated plant material, wherein said chemical is selected from the group consisting of detergents, organic solvents, and a combination thereof.

18. The method of claim 17, further comprising contacting said treated plant material with at least one enzyme capable of hydrolyzing lignocellulose.

19. The method of claim 18, wherein said plant material comprises at least one enzyme capable of hydrolyzing lignocellulose.

20. The method of claim 19, wherein said plant material comprises at least one plant that has been genetically engineered to produce at least one enzyme capable of hydrolyzing lignocellulose.

21. The method of claim 20, comprising incubating said plant material under conditions that allow production of said enzyme capable of hydrolyzing lignocellulose prior to contacting said plant material with said chemical.

22. The method of claim 17, wherein said substance is selected from the group consisting of an enzyme, a pharmaceutical, and a nutraceutical.

23. The method of claim 22, wherein said plant material comprises at least one plant that has been genetically engineered to produce said substance.
